(19) United States
(12) Patent Application Publication
(10) Pub. No.: US 2025/0121193 A1
(43) Pub. Date: Apr. 17, 2025

(54) SYSTEMS AND METHODS FOR MODULATING BRAIN ACTIVITY

(71) Applicant: Boston Scientific Neuromodulation Corporation, Valencia, CA (US)

(72) Inventors: Rafael Carbunaru, Valley Village, CA (US); Michael A. Moffitt, Solon, OH (US); Hemant Bokil, Cambridge, MA (US); Rosana Esteller, Santa Clarita, CA (US); Goran N. Marnfeldt, Valencia, CA (US); John Rivera, Oxnard, CA (US); Joseph M. Bocek, Seattle, WA (US)

(21) Appl. No.: 18/915,211

(22) Filed: Oct. 14, 2024

Related U.S. Application Data
(60) Provisional application No. 63/544,412, filed on Oct. 16, 2023.

(51) Int. Cl. A61N 1/36

(52) U.S. Cl. CPC A61N 1/36103; A61N 1/36132; A61N 1/36139

(57) ABSTRACT

A system may include a training system configured for training or improving a nervous system by responding to an event. The training system may include a neuromodulator configured for delivering neuromodulation to a nervous system, an event detector configured for detecting the event and sending a signal to the neuromodulator when the event is detected, and a timer configured for timing a predefined time period after receiving the signal. The neuromodulator may be configured to change neuromodulation to the nervous system at the end of the time period. The predefined time period may be a programmable time period in some embodiments.

### CLAIM OF PRIORITY

This application claims the benefit of U.S. Provisional Application No. 63/544,412, filed on Oct. 16, 2023, which is hereby incorporated by reference in its entirety.

### TECHNICAL FIELD

This document relates generally to medical systems, and more particularly, but not by way of limitation, to systems, devices, and methods for modulating brain connectivity for therapeutic purposes or functional enhancement.

### BACKGROUND

Medical devices may include therapy-delivery devices configured to deliver a therapy to a patient and/or monitors configured to monitor a patient condition via user input and/or sensor(s). For example, therapy-delivery devices for ambulatory patients may include wearable devices and implantable devices, and further may include, but are not limited to, stimulators (such as electrical, thermal, or mechanical stimulators) and drug delivery devices (such as an insulin pump). An example of a wearable device includes, but is not limited to, transcutaneous electrical neural stimulators (TENS), such as may be attached to glasses, an article of clothing, or a patch configured to be adhered to skin. Implantable stimulation devices may deliver electrical stimuli to treat various biological disorders, such spinal cord stimulators (SCS) to treat chronic pain, cortical and Deep Brain Stimulators (DBS) to treat motor and psychological disorders, Peripheral Nerve Stimulation (PNS) including Vagal Nerve Stimulation (VNS), Functional Electrical Stimulation (FES), and other neural stimulators to treat urinary incontinence, sleep apnea, shoulder subluxation, and the like.

As different parts of the brain tend to be responsible for different functions, treatments for various disorders conventionally attempt to stimulate the part of the brain that is responsible for a function associated with disorder. Thus, for example, one part of the brain responsible for movement may be stimulated to treat movement disorders and another part of the brain responsible for mood may be stimulated to treat psychological disorders. Although the nervous system (e.g., brain or other parts of the nervous system) has the ability to adapt or learn, conventional therapies are not designed to take advantage of this ability. What is needed is an improved system and method that may be used to encourage the nervous system to adapt to effectively treat the disorder.

### SUMMARY

Embodiments of the present subject matter are capable of using neural stimulation to train or to improve or enhance the brain to adapt to desirably respond to an event. For example, in addition or alternatively to training the brain, the intrinsic state dynamics of the brain may benefit from stimulating at certain epochs within a task. For instance, if one thinks of a stimulation paradigm for something like Freezing of gait, it may be that the appropriate way to stimulate is to stimulate at particular epochs in the patient's attempted gait and that stimulation at other epochs is detrimental. Not because of any training per se, but just because there is an intrinsic state dynamics that makes this type of stimulation necessary.

The system may be designed to enable a user to configure the system to train or improve (e.g., enhance) the brain (or other part of the nervous system) to appropriately respond to a variety of events, such as various motor events or psychological events. For example, reinforcement neural stimulation of a location in the brain may be delivered or a pulse pattern may be changed when a task is performed well, and/or an abatement neural stimulation of a location in the brain may be delivered when a task is not satisfactorily performed. An example of an abatement neural stimulation may include stopping the neural stimulation or changing a pulse pattern and/or burst pattern within the neural stimulation to negatively reinforce a poorly performed response (e.g., task) to an event. The reinforcement stimulation may function to encourage a learned behavior and the abatement neural stimulation may function to discourage learned behavior. Changes in stimulation may be spatial changes (e.g., location) temporal changes (e.g., pulse width, pulse frequency, duration or pulse number for a burst of pulses, burst frequency, and various pulse patterns and/or burst patterns). The time from the event to the stimulation change may be changed to provide a desired learned response to the event.

An example (e.g., “Example 1”) of a system may include a training system configured for training or improving a nervous system by responding to an event. The training system may include a neuromodulator configured for delivering neuromodulation to a nervous system, an event detector configured for detecting the event and sending a signal to the neuromodulator when the event is detected, and a timer configured for timing a predefined time period after receiving the signal. The neuromodulator may be configured to change neuromodulation to the nervous system at an end of the time period. The predefined time period may be a programmable time period in some embodiments.

In Example 2, the subject matter of Example 1 may optionally be configured such that the neuromodulator and the event detector have synched clocks for use to provide precise timing from a time when the event is detected to a time when the neuromodulation is changed.

In Example 3, the subject matter of any one or more of Examples 1-2 may optionally be configured such that the event detector includes at least one of: a user device configured for use to enter or determine an endpoint of or a period or a phase in a task based on time since the task was started or to provide a computer-based information corresponding to a computer-based task, at least one wearable sensor configured to sense a parameter indicative of a status of the task, or at least one sensor attached or integrated into an object used to perform the task.

In Example 4, the subject matter of any one or more of Examples 1-3 may optionally be configured such that the event detector is configured to use at least two conditions to detect a state, and to use the detected state as the event.

In Example 5, the subject matter of any one or more of Examples 1-4 may optionally be configured such that the event detector is configured to detect a stimulation channel activity as a surrogate for the event.

In Example 6, the subject matter of any one or more of Examples 1-5 may optionally be configured to further include a neuromodulation device configured to implement a first process to detect the event and to implement a second process to change the neuromodulation. The first process may be configured to send the signal to the second process.

In Example 7, the subject matter of any one or more of Examples 1-5 may optionally be configured to include wearable sensors. The wearable sensors may be configured for use to detect the event. The wearable sensors may be configured to send the signal to the neuromodulator.

In Example 8, the subject matter of any one or more of Examples 1-7 may optionally be configured to include implantable sensors. The implantable sensors may be configured for use to detect the event. The implantable sensors may be configured to send the signal to the neuromodulator.

In Example 9, the subject matter of any one or more of Examples 1-8 may optionally be configured to further include a user device configured to receive input for use to detect the event.

In Example 10, the subject matter of any one or more of Examples 1-9 may optionally be configured such that the signal sent to the neuromodulator includes information used by the neuromodulator to determine at least one of: the predefined time period; or information used to change the neuromodulation.

In Example 11, the subject matter of any one or more of Examples 1-10 may optionally be configured to provide user selection of the period of time from two or more of: 1's of milliseconds, 10's of milliseconds, 100's of milliseconds, 1's of seconds, 10's of seconds, minutes or hours. Some embodiments may allow a user to program specific times within these ranges.

In Example 12, the subject matter of any one or more of Examples 1-11 may optionally be configured such that the neuromodulator is configured to change neuromodulation to the nervous system by turning stimulation on or off, changing at least one stimulation value, changing a number of stimulation pulses, changing a stimulation pattern, or changing an electrode fractionalization used to deliver the neuromodulation.

In Example 13, the subject matter of any one or more of Examples 1-12 may optionally be configured to further include a training evaluator configured to monitor an observed response of the nervous system to the event and comparing the observed response to a desired response, and an optimizer configured to implement an optimization algorithm to improve the training or the improving of the nervous system to cause the observed response to the event to be closer to the desired response.

In Example 14, the subject matter of Example 13 may optionally be configured such that the optimizer is configured to identify an adjustment to a set of neuromodulation parameters, an adjustment to the predefined period of time for changing the neuromodulation, or an adjustment to at least one event detection criterion used to detect the event.

In Example 15, the subject matter of any one or more of Examples 1-14 may optionally be configured to further include a deep brain stimulator (DBS) configured for delivering neuromodulation to a brain, a spinal cord stimulator (SCS) configured for delivering neuromodulation to neural targets in or near the spinal cord, or a peripheral nerve stimulator (PNS) configured for delivering neuromodulation to a neural target in a peripheral nervous system.

Example 16 includes subject matter (such as a method, means for performing acts, machine readable medium including instructions that when performed by a machine cause the machine to perform acts, or an apparatus to perform). The subject matter may include training or improving a nervous system by responding to an event. The nervous system may be trained by detecting the event using at least one event detection criterion, sending a signal to a neuromodulator when an event is detected, waiting a predefined time period after receiving the signal, and then at the end of the predefined time period changing neuromodulation delivered by the neuromodulator to the nervous system. The nervous system has plasticity for learning to respond to the event, and changed neuromodulation at the end of the time period promotes learning by the nervous system to respond to the event. The predefined time period may be a programmable time period in some embodiments.

In Example 17, the subject matter of Example 16 may optionally be configured such that the detecting the event includes determining an endpoint of or a period or a phase in a task based on time since the task was started, a computer-based information corresponding to a computer-based task, at least one wearable sensor configured to sense a parameter indicative of a status of the task, or at least one sensor attached or integrated into an object used to perform the task.

In Example 18, the subject matter of any one of Examples 16-17 may optionally be configured such that the detecting the event includes receiving user input indicative of the event.

In Example 19, the subject matter of any one of Examples 16-18 may optionally be configured such that the detecting the event includes detecting a state indicative of the event.

In Example 20, the subject matter of Example 19 may optionally be configured such that the detecting the event includes detecting two or more conditions and using the detected two or more conditions to detect the state.

In Example 21, the subject matter of any one of Examples 19-20 may optionally be configured such that the detecting the event includes detecting a stimulation channel activity as a surrogate for the event.

In Example 22, the subject matter of any one of Examples 16-21 may optionally be configured such that the event includes a trigger event for implementing a command sequence of one or more preset commands. The subject matter may further include implementing the command sequence in response to the detected event.

In Example 23, the subject matter of any one of Examples 16-22 may optionally be configured such that the sending the signal to the neuromodulator includes sending the signal from one process within the neuromodulator used to detect the event to another process within the neuromodulator used to change the neuromodulation.

In Example 24, the subject matter of any one of Examples 16-23 may optionally be configured such that the sending the signal includes wirelessly sending the signal to the neuromodulator from another device.

In Example 25, the subject matter of any one of Examples 16-24 may optionally be configured such that the signal sent to the neuromodulation includes information used by the neuromodulator to determine at least one of the predefined time period or information used to change the neuromodulation.

In Example 26, the subject matter of any one of Examples 16-25 may optionally be configured to further include determining timing from available timing selections for sending the signal based on the event and a target of the delivered neuromodulation. The available timing selections may include two or more of: 1's of milliseconds, 10's of milliseconds, 100's of milliseconds, 1's of seconds, 10's of seconds, minutes or hours.

In Example 27, the subject matter of any one of Examples 16-26 may optionally be configured to further include turning stimulation on or off, changing at least one stimulation parameter value, changing a number of stimulation pulses, changing a stimulation pattern, or changing an electrode fractionalization used to deliver the neuromodulation.

In Example 28, the subject matter of any one of Examples 16-27 may optionally be configured such that the training or the improving the nervous system to respond to the event includes training or improving a brain of a patient to recall events associated with positive feelings or to emphasize neutral feeling.

In Example 29, the subject matter of any one of Examples 16-28 may optionally be configured to further include evaluating the training or the improving by monitoring an observed response of the nervous system to the event and comparing the observed response to a desired response and implementing an optimizer algorithm to improve the training of the nervous system to cause the observed response to the event to be closer to the desired response.

In Example 30, the subject matter of any one of Examples 16-28 may optionally be configured such that the implementing the optimizer algorithm includes adjusting a set of neuromodulation parameters, adjusting the predefined period of time for changing the neuromodulation, or changing one or more of at least one event detection criteria used to detect the event.

Example 31 includes subject matter that includes non-transitory machine-readable medium including instructions, which when executed by a machine, cause the machine to perform a method. The method performed by the machine may include training or improving a nervous system by responding to an event. The nervous system may be trained by detecting the event using at least one event detection criterion, sending a signal to a neuromodulator when the event is detected, waiting a predefined time period after receiving the signal, and then at the end of the predefined time period changing neuromodulation delivered by the neuromodulator to the nervous system. The nervous system has plasticity for learning to respond to the event, and changed neuromodulation at an end of the time period promotes learning by the nervous system to respond to the event. The predefined time period may be a programmable time period in some embodiments. In further examples, the subject matter of Example 31 may be configured such that the method performed by the machine may include any of the subject matter recited in Examples 17-30.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, an electrical stimulation system, which may be used to deliver DBS.

FIG. 2 illustrates, by way of example and not limitation, an implantable pulse generator (IPG) in a DBS system.

FIGS. 3A-3B illustrate, by way of example and not limitation, leads that may be coupled to the IPG to deliver electrostimulation such as DBS.

FIG. 4 illustrates, by way of example and not limitation, a computing device for programming or controlling the operation of an electrical stimulation system.

FIG. 5 illustrates, by way of example and not limitation, a more generalized example of a medical system that includes a medical device and a processing system.

FIG. 6 illustrates, by way of example, an example of an electrical therapy-delivery system.

FIG. 7 illustrates, by way of example and not limitation, a monitoring system and/or the electrical therapy-delivery system of FIG. 6, implemented using an IMD.

FIGS. 8A-8C illustrate, by way of example and not limitation, a nervous system response, neuromodulation training for training or improving the nervous system based on an event or state within the nervous system (e.g., brain), and a trained or improved nervous system to provide the desired response.

FIG. 9 illustrates, by way of example and not limitation, a training system configured for training the nervous system to respond to an event.

FIG. 10 illustrates, by way of example and not limitation, a neuromodulation device configured with a training system.

FIG. 11 illustrates, by way of example and not limitation, a training system distributed among a neuromodulation device and an external system.

FIG. 12 illustrates, by way of example and not limitation, a process for training a nervous system to respond to an event.

FIG. 13 illustrates, by way of example and not limitation, a process for training a nervous system with an optimizer.

FIG. 14 illustrates, by way of example and not limitation, timing interaction between an initiator and a receiver to implement a training session.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized, and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

It is desirable to be able to sense and close the loop with stimulation. However, sensing and interpreting the brain is difficult. Various embodiments provide a platform for therapeutically exploiting the plasticity of the brain or other neuromodulation targets. Stimulation change may be used to modulate brain performance for therapeutic purposes. The stimulation may be delivered at a time when the “state” of the brain corresponds to a specified task-based event. Complex tasks can often be thought of as comprised of different stages. Therefore, “states” may not correspond to a “specified task-based event” but may rather be epochs in a complex task.

The brain learns by integrating many inputs and specifically the timing of those inputs. Among other things, this learning includes modifying its own processing behavior in response to future inputs. This property allows us to learn and improve performance in a great many things. Although the learning sometimes starts at a cognitive level, brain behavior is changed to enable subsequent performance at a sub-cognitive level. Examples include motor tasks such as learning to type or learning a sport-related motion until it becomes second nature. Other examples include sensory, processing, and reacting tasks such as learning a language. Other examples include affective responses such as generating a feeling in response to scary music or happy music where learning is in connection to prior experience.

A significant trigger of brain plasticity is the timing of events which are reflected as neural inputs, and events that happen during a given brain “state”. Stimulation (and inhibition) of midbrain dopamine neurons during a reaching task has been investigated. Stimulating during the reach was found to degrade performance but stimulating between reaches does not. The effect of inhibition also depends on timing (Alexandra Bova, Matt Gaidica, Amy Hurst, Yoshiko Iwai, Julia Hunter, Daniel K Leventhal (2020). Precisely timed dopamine signals establish distinct kinematic representations of skilled movements eLife 9:e61591). This suggests that turning stimulation OFF at certain epochs may be important. DBS (continuous and adaptive to beta band power) during a random moving dot direction detection task has been investigated. DBS during certain narrow time windows has been found to impair task performance (Herz, D. M., Bange, M., Gonzalez-Escamilla, G. et al. Dynamic control of decision and movement speed in the human basal ganglia. Nat Commun 13, 7530 (2022). https://doi.org/10.1038/s41467-022-35121-8). This suggests that turning stimulation OFF triggered to external cues may be important. It has been found that the effect of transcranial magnetic stimulation (TMS) on memory depends on timing of stimulation (better effect when stimulation is during the memory retention phase rather than during the test phase) (Luber B, Kinnunen L H, Rakitin B C, Ellsasser R, Stern Y, Lisanby S H. Facilitation of performance in a working memory task with rTMS stimulation of the precuneus: frequency- and time-dependent effects. Brain Res. 2007 Jan. 12; 1128(1):120-9. doi: 10.1016/j.brainres.2006.10.011. Epub 2006 Nov. 20. PMID: 17113573). This suggests that stimulation should preferentially be turned on during these periods. Spacing appears to be important in learning. (Santoro, Helen. “The Neuroscience Behind the Spacing Effect.” BrainFacts.Org, 4 Mar. 2021, www.brainfacts.org/thinking-sensing-and-behaving/learning-and-memory/2021/the-neuroscience-behind-the-spacing-effect-030421. Accessed 8 Aug. 2023.) The superiority of spaced learning or training may be attributable to various underlying mechanisms of action. (Smolen, P., Zhang, Y. & Byrne, J. The right time to learn: mechanisms and optimization of spaced learning. Nat Rev Neurosci 17, 77-88 (2016). https://doi.org/10.1038/nrn.2015.18.)

Various embodiments of the present subject matter provide systems and methods that enable coordination of output changes (e.g., neurostimulation or neuromodulation changes) with motor events or other events consciously translated to motor events. Examples of motor events may include a motor task, a cognitive task translated to a motor task (e.g., pressing a button to indicate completion of a cognitive task), an affective or mood change translated to a motor task (e.g., providing an input to an app upon feeling something), a sensory change translated to a motor task (e.g., pressing a button upon hearing or seeing something). Further, the action that the event triggers, or does not trigger, might also be state dependent such as a nerve or brain state determined by biopotential recordings, brain state determined by patient or care-giver inputs, physiological state of an aspect of the body, external environment state which may be linked to brain state inasmuch as the patient has awareness of the environmental state through natural senses. They may also be a function of the “learning state” of the subject as inferred from a model of learning. For example, in repetitive tasks the subject's performance may improve over time for “natural” reasons and the goal of our stimulation may be to improve the learning rate rather than the performance itself. It may also be possible to use stimulation to enhance ‘mindfulness’ based pain control, whereby self-regulation of brain functions (cognitive, affective pain matrix components) may be enhanced/efficiently learned by combining stimulation with biofeedback, for example. The systems and methods provided by the subject matter may enable clinicians and scientists to take advantage of the plastic properties of the brain (i.e., to remodel brain behavior) for therapeutic benefit or performance enhancement.

Due to neuropathologies such as but not limited to neurodegenerative disease or injury, or due to negative environment inputs that affect neurodevelopment such as but not limited to traumatic experiences and choices leading to addiction, the nervous system enters states that constitute neurological dysfunction or disorder and affect patients adversely. In these cases, there is a need to be able to retrain the brain such that these pathological states or activations are diminished in magnitude, avoided, or mitigated, etc., and/or conversely, that positive states are developed, learned, or engaged more often for therapeutic purposes.

To accomplish the above, some embodiments of the present subject matter may have the ability to detect events and change neuromodulation is response to event detections. Precise timing of the response with respect to the event may create the desired learning by the brain or nervous system. External or implantable sensors for automatic detection/triggering, and/or devices for manually communicating an event may be used for event detection and/or communication. The system may assess brain state using various types of inputs such as but not limited to biopotential based, accelerometer based, sensed signatures or inputs during a controlled task, wearables, physiological measurement devices, and processing of the aforementioned, so that response to events can be state dependent. Of note, assessment of brain state can be used to assess any change in brain state, or the time course of change in brain state, during the course of therapy. This may be used to assess and compare the benefits of different therapy options, or the effects of therapy being withheld (e.g., for controlled studies). Some features of various embodiments relate to events, state information, stimulation outputs to the nervous system, coordination and timing of the stimulation change to the detected event, and closed loop optimization of event triggered modulations.

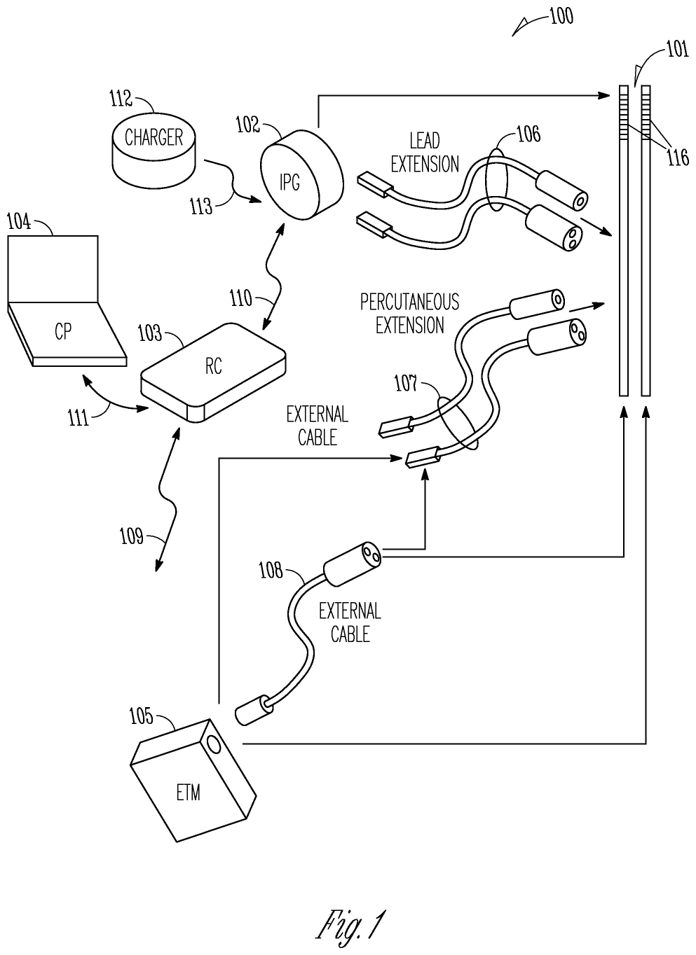

FIG. 1 illustrates, by way of example and not limitation, an electrical stimulation system 100, which may be used to deliver DBS. The electrical stimulation system 100 may generally include a one or more (illustrated as two) of implantable neuromodulation leads 101, a waveform generator such as an implantable pulse generator (IPG) 102, an external remote controller (RC) 103, a clinician programmer (CP) 104, and an external trial modulator (ETM) 105. The IPG 102 may be physically connected via one or more percutaneous lead extensions 106 to the neuromodulation lead(s) 101, which carry a plurality of electrodes 116. The electrodes, when implanted in a patient, form an electrode arrangement. As illustrated, the neuromodulation leads 101 may be percutaneous leads with the electrodes arranged in-line along the neuromodulation leads or about a circumference of the neuromodulation leads. Any suitable number of neuromodulation leads can be provided, including only one, as long as the number of electrodes is greater than two (including the IPG case function as a case electrode) to allow for lateral steering of the current. Other types of leads may be used. The IPG 102 includes pulse generation circuitry that delivers electrical modulation energy in the form of a pulsed electrical waveform (i.e., a temporal series of electrical pulses) to the electrodes in accordance with a set of modulation parameters.

The ETM 105 may also be physically connected via the percutaneous lead extensions 107 and external cable 108 to the neuromodulation lead(s) 101. The ETM 105 may have similar pulse generation circuitry as the IPG 102 to deliver electrical modulation energy to the electrodes in accordance with a set of modulation parameters. The ETM 105 is a non-implantable device that may be used on a trial basis after the neuromodulation leads 101 have been implanted and prior to implantation of the IPG 102, to test the responsiveness of the modulation that is to be provided. The ETM 105 may be used for situations where a brief period of therapy is suitable to achieve the desired effects. Functions described herein with respect to the IPG 102 can likewise be performed with respect to the ETM 105.

The RC 103 may be used to telemetrically control the ETM 105 via a bi-directional RF communications link 109. The RC 103 may be used to telemetrically control the IPG 102 via a bi-directional RF communications link 110. Such control allows the IPG 102 to be turned ON or OFF and to be programmed with different modulation parameter sets. The IPG 102 may also be operated to modify the programmed modulation parameters to actively control the characteristics of the electrical modulation energy output by the IPG 102. A clinician may use the CP 104 to program modulation parameters into the IPG 102 and ETM 105 in the operating room and in follow-up sessions.

The CP 104 may indirectly communicate with the IPG 102 or ETM 105, through the RC 103, via an IR communications link 111 or another link. The CP 104 may directly communicate with the IPG 102 or ETM 105 via an RF communications link or other link (not shown). The clinician detailed modulation parameters provided by the CP 104 may also be used to program the RC 103, so that the modulation parameters can be subsequently modified by operation of the RC 103 in a stand-alone mode (i.e., without the assistance of the CP 104). Various devices may function as the CP 104. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 104. Alternatively, such programming methodologies can be performed using firmware or hardware. In any event, the CP 104 may actively control the characteristics of the electrical modulation generated by the IPG 102 to allow the desired parameters to be determined based on patient feedback or other feedback and for subsequently programming the IPG 102 with the desired modulation parameters. To allow the user to perform these functions, the CP 104 may include user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g., CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical modulation energy output by the neuromodulation leads, and select and program the IPG with modulation parameters, including electrode selection, in both a surgical setting and a clinical setting. The external device(s) (e.g., CP and/or RC) may be configured to communicate with other device(s), including local device(s) and/or remote device(s). For example, wired and/or wireless communication may be used to communicate between or among the devices.

An external charger 112 may be a portable device used to transcutaneously charge the IPG 102 via a wireless link such as an inductive link 113. Once the IPG 102 has been programmed, the IPG 102 may function as programmed without the RC 103 or CP 104 being present. It is noted that some IPGs do not require charging, as some are manufactured with primary batteries with sufficient capacity to provide therapy over a clinically useful duration without recharging.

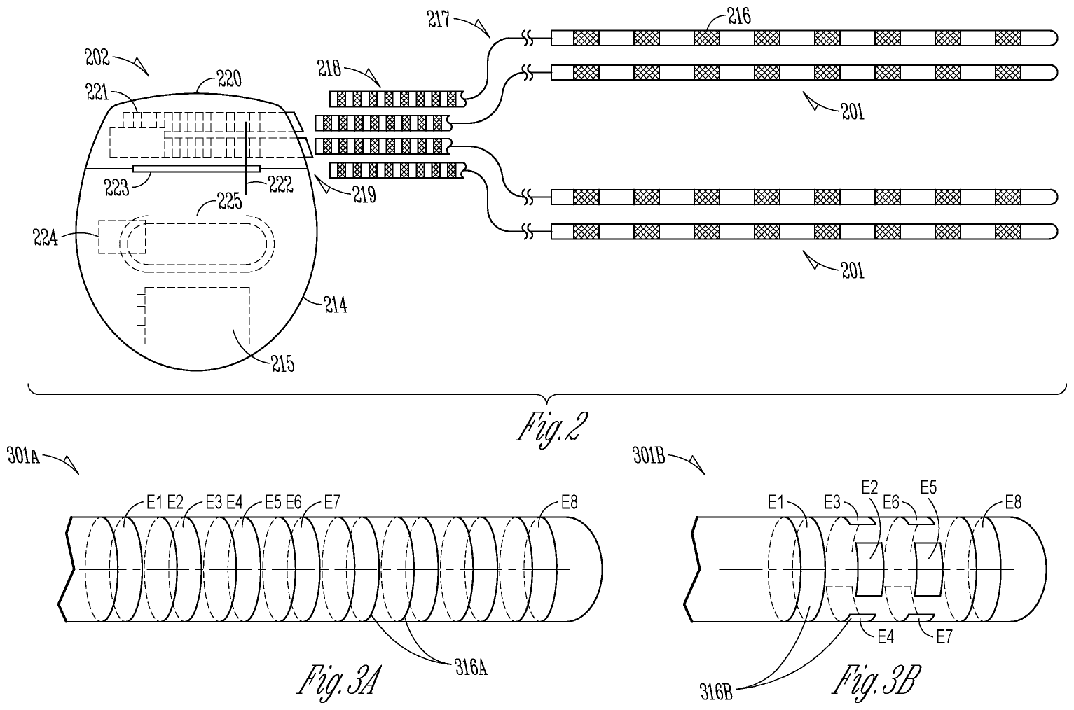

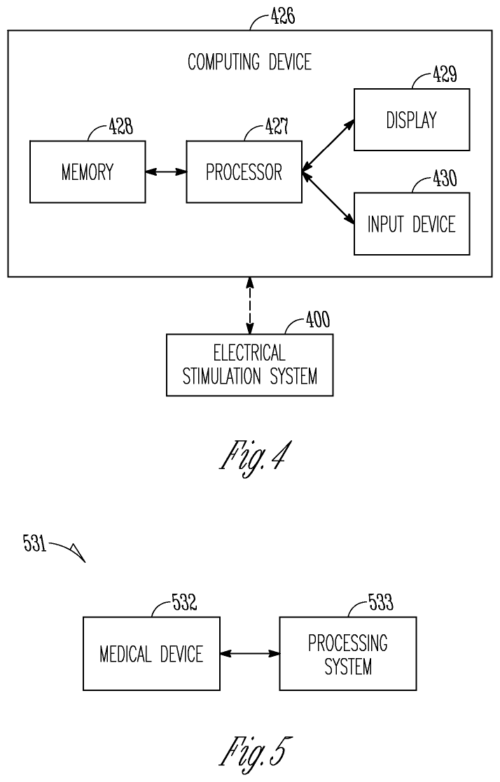

FIG. 2 illustrates, by way of example and not limitation, an IPG 202 in a DBS system. The IPG 202, which is an example of the IPG 102 of the electrical stimulation system 100 as illustrated in FIG. 1, may include a biocompatible device case 214 that holds the circuitry and a battery 215 for providing power for the IPG 202 to function, although the IPG 202 can also lack a battery and can be wirelessly powered by an external source. The IPG 202 may be coupled to one or more leads, such as leads 201 as illustrated herein. The leads 201 can each include a plurality of electrodes 216 for delivering electrostimulation energy, recording electrical signals, or both. In some examples, the leads 201 can be rotatable so that the electrodes 216 can be aligned with the target neurons after the neurons have been located such as based on the recorded signals. The electrodes 216 can include one or more ring electrodes, and/or one or more sets of segmented electrodes (or any other combination of electrodes), examples of which are discussed below with reference to FIGS. 3A and 3B.

The leads 201 can be implanted near or within the desired portion of the body to be stimulated. In an example of operations for DBS, access to the desired position in the brain can be accomplished by drilling a hole in the patient's skull or cranium with a cranial drill (commonly referred to as a burr), and coagulating and incising the dura mater, or brain covering. A lead can then be inserted into the cranium and brain tissue with the assistance of a stylet (not shown). The lead can be guided to the target location within the brain using, for example, a stereotactic frame and a microdrive motor system. In some examples, the microdrive motor system can be fully or partially automatic. The microdrive motor system may be configured to perform actions such as inserting, advancing, rotating, or retracting the lead.

Lead wires 217 within the leads may be coupled to the electrodes 216 and to proximal contacts 218 insertable into lead connectors 219 fixed in a header 220 on the IPG 202, which header can comprise an epoxy for example. Alternatively, the proximal contacts 218 may connect to lead extensions (not shown) which are in turn inserted into the lead connectors 219. Once inserted, the proximal contacts 218 connect to header contacts 221 within the lead connectors 219, which are in turn coupled by feedthrough pins 222 through a case feedthrough 223 to stimulation circuitry 224 within the case 214. The type and number of leads, and the number of electrodes, in an IPG is application specific and therefore can vary.

The IPG 202 can include an antenna 225 allowing it to communicate bi-directionally with a number of external devices. The antenna 225 may be a conductive coil within the case 214, although the coil of the antenna 225 may also appear in the header 220. When the antenna 225 is configured as a coil, communication with external devices may occur using near-field magnetic induction. The IPG may also include a Radio-Frequency (RF) antenna. The RF antenna may comprise a patch, slot, or wire, and may operate as a monopole or dipole, and preferably communicates using far-field electromagnetic waves, and may operate in accordance with any number of known RF communication standards, such as Bluetooth, Zigbee, WiFi, Medical Implant Communication System (MICS), and the like.

In a DBS application, as is useful in the treatment of tremor in Parkinson's disease for example, the IPG 202 is typically implanted under the patient's clavicle (collarbone). The leads 201 (which may be extended by lead extensions, not shown) can be tunneled through and under the neck and the scalp, with the electrodes 216 implanted through holes drilled in the skull and positioned for example in the subthalamic nucleus (STN) and the pedunculopontine nucleus (PPN) in each brain hemisphere. The IPG 202 can also be implanted underneath the scalp closer to the location of the electrodes' implantation. The leads 201, or the extensions, can be integrated with and permanently connected to the IPG 202 in other solutions.

Stimulation in IPG 202 is typically provided by pulses each of which may include one phase or multiple phases. For example, a monopolar stimulation current can be delivered between a lead-based electrode (e.g., one of the electrodes 216) and a case electrode. A bipolar stimulation current can be delivered between two lead-based electrodes (e.g., two of the electrodes 216). Stimulation parameters typically include current amplitude (or voltage amplitude), frequency, pulse width of the pulses or of its individual phases; electrodes selected to provide the stimulation; polarity of such selected electrodes, i.e., whether they act as anodes that source current to the tissue, or cathodes that sink current from the tissue. Each of the electrodes can either be used (an active electrode) or unused (OFF). When the electrode is used, the electrode can be used as an anode or cathode and carry anodic or cathodic current. In some architectures, electrodes of the same polarity can deliver distinct amounts of current simultaneously using multiple electrical sources, to provide greater control of the electric field. In some instances, an electrode might be an anode for a period of time and a cathode for a period of time (e.g., when multiple phases are used, for example, for charge recovery or other purposes). These and possibly other stimulation parameters taken together comprise a stimulation program that the stimulation circuitry 224 in the IPG 202 can execute to provide therapeutic stimulation to a patient.

In some examples, a measurement device coupled to the muscles or other tissue stimulated by the target neurons, or a unit responsive to the patient or clinician, can be coupled to the IPG 202 or microdrive motor system. The measurement device, user, or clinician can indicate a response by the target muscles or other tissue to the stimulation or recording electrode(s) to further identify the target neurons and facilitate positioning of the stimulation electrode(s). For example, if the target neurons are directed to a muscle experiencing tremors, a measurement device can be used to observe the muscle and indicate changes in, for example, tremor frequency or amplitude in response to stimulation of neurons. Alternatively, the patient or clinician can observe the muscle and provide feedback.

FIGS. 3A-3B illustrate, by way of example and not limitation, leads that may be coupled to the IPG to deliver electrostimulation such as DBS. FIG. 3A shows a lead 301A with electrodes 316A disposed at least partially about a circumference of the lead 301A. The electrodes 316A may be located along a distal end portion of the lead. As illustrated herein, the electrodes 316A are ring electrodes that span 360 degrees about a circumference of the lead 301. A ring electrode allows current to project equally in every direction from the position of the electrode, and typically does not enable stimulus current to be directed from only a particular angular position or a limited angular range around of the lead. A lead which includes only ring electrodes may be referred to as a non-directional lead.

FIG. 3B shows a lead 301B with electrodes 316B including ring electrodes such as E1 at a proximal end and E8 at the distal end. Additionally, the lead 301 also include a plurality of segmented electrodes (also known as split-ring electrodes). For example, a set of segmented electrodes E2, E3, and E4 are around the circumference at a longitudinal position, each spanning less than 360 degrees around the lead axis. In an example, each of electrodes E2, E3, and E4 spans 90 degrees, with each being separated from the others by gaps of 30 degrees. Another set of segmented electrodes E5, E6, and E7 are located around the circumference at another longitudinal position different from the segmented electrodes E2, E3 and E4. Segmented electrodes such as E2-E7 can direct stimulus current to a selected angular range around the lead.

Segmented electrodes can typically provide superior current steering than ring electrodes because target structures in DBS or other stimulation are not typically symmetric about the axis of the distal electrode array. Instead, a target may be located on one side of a plane running through the axis of the lead. Through the use of a radially segmented electrode array, current steering using multiple electrical sources can be performed not only along a length of the lead but also around a circumference of the lead. This provides precise three-dimensional targeting and delivery of the current stimulus to neural target tissue, while potentially avoiding stimulation of other tissue. In some examples, segmented electrodes can be together with ring electrodes. A lead which includes at least one or more segmented electrodes may be referred to as a directional lead. In an example, all electrodes on a directional lead can be segmented electrodes. In another example, there can be different numbers of segmented electrodes at different longitudinal positions.

Segmented electrodes may be grouped into sets of segmented electrodes, where each set is disposed around a circumference at a particular longitudinal location of the directional lead. The directional lead may have any number of segmented electrodes in a given set of segmented electrodes. By way of example and not limitation, a given set may include any number between two to sixteen segmented electrodes. In an example, all sets of segmented electrodes may contain the same number of segmented electrodes. In another example, one set of the segmented electrodes may include a different number of electrodes than at least one other set of segmented electrodes.

The segmented electrodes may vary in size and shape. In some examples, the segmented electrodes are all of the same size, shape, diameter, width or area or any combination thereof. In some examples, the segmented electrodes of each circumferential set (or even all segmented electrodes disposed on the lead) may be identical in size and shape. The sets of segmented electrodes may be positioned in irregular or regular intervals along a length of the lead

FIG. 4 illustrates, by way of example and not limitation, a computing device 426 for programming or controlling the operation of an electrical stimulation system 400. The computing device 426 may include a processor 427, a memory 428, a display 429, and an input device 430. Optionally, the computing device 426 may be separate from and communicatively coupled to the electrical stimulation system 400, such as system 100 in FIG. 1. Alternatively, the computing device 426 may be integrated with the electrical stimulation system 100, such as part of the IPG 102, RC 103, CP 104, or ETM 105 illustrated in FIG. 1.

The computing device 426, also referred to as a programming device, can be a computer, tablet, mobile device, or any other suitable device for processing information. The computing device 426 can be local to the user or can include components that are non-local to the computer including one or both of the processor 427 or memory 428 (or portions thereof). For example, the user may operate a terminal that is connected to a non-local processor or memory. The functions associated with the computing device 426 may be distributed among two or more devices, such that there may be two or more memory devices performing memory functions, two or more processors performing processing functions, two or more displays performing display functions, and/or two or more input devices performing input functions. In some examples, the computing device 406 can include a watch, wristband, smartphone, or the like. Such computing devices can wirelessly communicate with the other components of the electrical stimulation system, such as the CP 104, RC 103, ETM 105, or IPG 102 illustrated in FIG. 1. The computing device 426 may be used for gathering patient information, such as general activity level or present queries or tests to the patient to identify or score pain, depression, stimulation effects or side effects, cognitive ability, or the like. In some examples, the computing device 426 may prompt the patient to take a periodic test (for example, every day) for cognitive ability to monitor, for example, Alzheimer's disease. In some examples, the computing device 426 may detect, or otherwise receive as input, patient clinical responses to electrostimulation such as DBS, and determine or update stimulation parameters using a closed-loop algorithm based on the patient clinical responses. Examples of the patient clinical responses may include physiological signals (e.g., heart rate) or motor parameters (e.g., tremor, rigidity, bradykinesia). The computing device 426 may communicate with the CP 104, RC 103, ETM 105, or IPG 102 and direct the changes to the stimulation parameters to one or more of those devices. In some examples, the computing device 426 can be a wearable device used by the patient only during programming sessions. Alternatively, the computing device 426 can be worn all the time and continually or periodically adjust the stimulation parameters. In an example, a closed-loop algorithm for determining or updating stimulation parameters can be implemented in a mobile device, such as a smartphone, that is connected to the IPG or an evaluating device (e.g., a wristband or watch). These devices can also record and send information to the clinician.

The processor 427 may include one or more processors that may be local to the user or non-local to the user or other components of the computing device 426. A stimulation setting (e.g., parameter set) includes an electrode configuration and values for one or more stimulation parameters. The electrode configuration may include information about electrodes (ring electrodes and/or segmented electrodes) selected to be active for delivering stimulation (ON) or inactive (OFF), polarity of the selected electrodes, electrode locations (e.g., longitudinal positions of ring electrodes along the length of a non-directional lead, or longitudinal positions and angular positions of segmented electrodes on a circumference at a longitudinal position of a directional lead), stimulation modes such as monopolar pacing or bipolar pacing, etc. The stimulation parameters may include, for example, current amplitude values, current fractionalization across electrodes, stimulation frequency, stimulation pulse width, etc.

The processor 427 may identify or modify a stimulation setting through an optimization process until a search criterion is satisfied, such as until an optimal, desired, or acceptable patient clinical response is achieved. Electrostimulation programmed with a setting may be delivered to the patient, clinical effects (including therapeutic effects and/or side effects, or motor symptoms such as bradykinesia, tremor, or rigidity) may be detected, and a clinical response may be evaluated based on the detected clinical effects. When actual electrostimulation is administered, the settings may be referred to as tested settings, and the clinical responses may be referred to as tested clinical responses. In contrast, for a setting in which no electrostimulation is delivered to the patient, clinical effects may be predicted using a computational model based at least on the clinical effects detected from the tested settings, and a clinical response may be estimated using the predicted clinical effects. When no electrostimulation is delivered the settings may be referred to as predicted or estimated settings, and the clinical responses may be referred to as predicted or estimated clinical responses.

In various examples, portions of the functions of the processor 427 may be implemented as a part of a microprocessor circuit. The microprocessor circuit can be a dedicated processor such as a digital signal processor, application specific integrated circuit (ASIC), microprocessor, or other type of processor for processing information. Alternatively, the microprocessor circuit can be a processor that can receive and execute a set of instructions of performing the functions, methods, or techniques described herein.

The memory 428 can store instructions executable by the processor 427 to perform various functions including, for example, determining a reduced or restricted electrode configuration and parameter search space (also referred to as a “restricted search space”), creating or modifying one or more stimulation settings within the restricted search space, etc. The memory 428 may store the search space, the stimulation settings including the “tested” stimulation settings and the “predicted” or “estimated” stimulation settings, clinical effects (e.g., therapeutic effects and/or side effects) and clinical responses for the settings.

The memory 428 may be a computer-readable storage media that includes, for example, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information, and which can be accessed by a computing device.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, Bluetooth, near field communication, and other wireless media.

The display 429 may be any suitable display or presentation device, such as a monitor, screen, display, or the like, and can include a printer. The display 429 may be a part of a user interface configured to display information about stimulation settings (e.g., electrode configurations and stimulation parameter values and value ranges) and user control elements for programming a stimulation setting into an IPG.

The input device 430 may be, for example, a keyboard, mouse, touch screen, track ball, joystick, voice recognition system, or any combination thereof, or the like. Another input device 430 may be a camera from which the clinician can observe the patient. Yet another input device 430 may a microphone where the patient or clinician can provide responses or queries.

The electrical stimulation system 400 may include, for example, any of the components illustrated in FIG. 1. The electrical stimulation system 400 may communicate with the computing device 426 through a wired or wireless connection or, alternatively or additionally, a user can provide information between the electrical stimulation system 400 and the computing device 426 using a computer-readable medium or by some other mechanism.

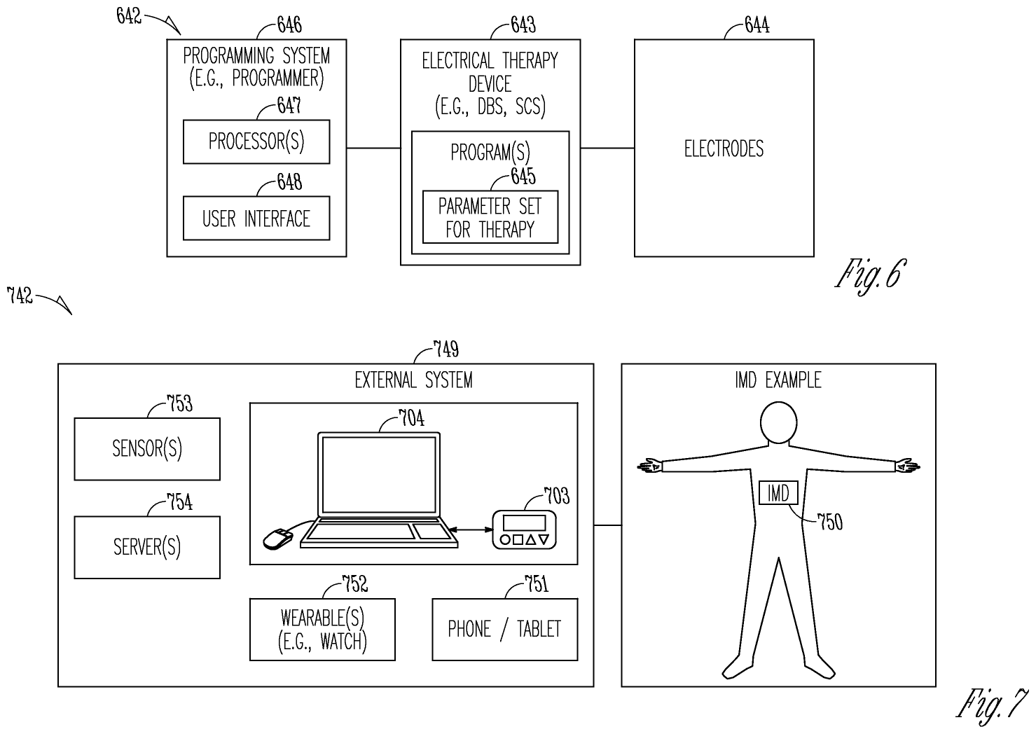

FIG. 5 illustrates, by way of example and not limitation, a more generalized example of a medical system 531 that includes a medical device 532 and a processing system 533. For example, the electrical stimulation system 400 of FIG. 4 may be a more specific example of the medical device 532 of FIG. 5, and computing device 426 of FIG. 4 may be a more specific example of the processing system 533 of FIG. 5. The medical device may be configured to provide sensing functions and/or therapy functions. For example, the medical device may include a device configured to use a parameter set to deliver an electrical stimulation therapy. The medical device may be an implantable medical device such as an implantable neurostimulator. The implantable medical device may be configured to deliver SCS or DBS therapy. The medical device may include more than one medical device. The processing system may be within a single device or may be a distributed system across two or more devices including local and/or remote systems. According to various embodiments, the medical system may include at least one medical device configured to treat a condition by delivering a therapy to a patient.

FIG. 6 illustrates, by way of example, an example of an electrical therapy-delivery system. The illustrated system 642 may be a more specific example of the system illustrated in FIG. 5 or form a portion of the system illustrated in FIG. 5. The illustrated system 642 includes an electrical therapy device 643 configured to deliver an electrical therapy to electrodes 644 to treat a condition in accordance with a programmed parameter set 645 for the therapy. The system 642 may include a programming system 646, which may function as at least a portion of a processing system, that may include one or more processors 647 and a user interface 648. The programming system 646 may be used to program and/or evaluate the parameter set(s) used to deliver the therapy. The illustrated system 642 may be a DBS system. In some embodiments, the illustrated system 642 may include an SCS system to treat pain and/or a system for monitoring pain. By way of example, a therapeutic goal for conventional SCS programming may be to maximize stimulation (i.e., recruitment) of the dorsal column (DC) fibers that run in the white matter along the longitudinal axis of the spinal cord and minimal stimulation of other fibers that run perpendicular to the longitudinal axis of the spinal cord (e.g., dorsal root fibers).

A therapy may be delivered according to a parameter set. The parameter set may be programmed into the device to deliver the specific therapy using specific values for a plurality of therapy parameters. For example, the therapy parameters that control the therapy may include pulse amplitude, pulse frequency, pulse width, and electrode configuration (e.g., selected electrodes, polarity and fractionalization). The parameter set includes specific values for the therapy parameters. The number of electrodes available combined with the ability to generate a variety of complex electrical waveforms (e.g., pulses), presents a huge selection of modulation parameter sets to the clinician or patient. For example, if the neuromodulation system to be programmed has sixteen electrodes, millions of modulation parameter sets may be available for programming into the neuromodulation system. To facilitate such selection, the clinician generally programs the modulation parameters sets through a computerized programming system to allow the optimum modulation parameters to be determined based on patient feedback or other means and to subsequently program the desired modulation parameter sets.

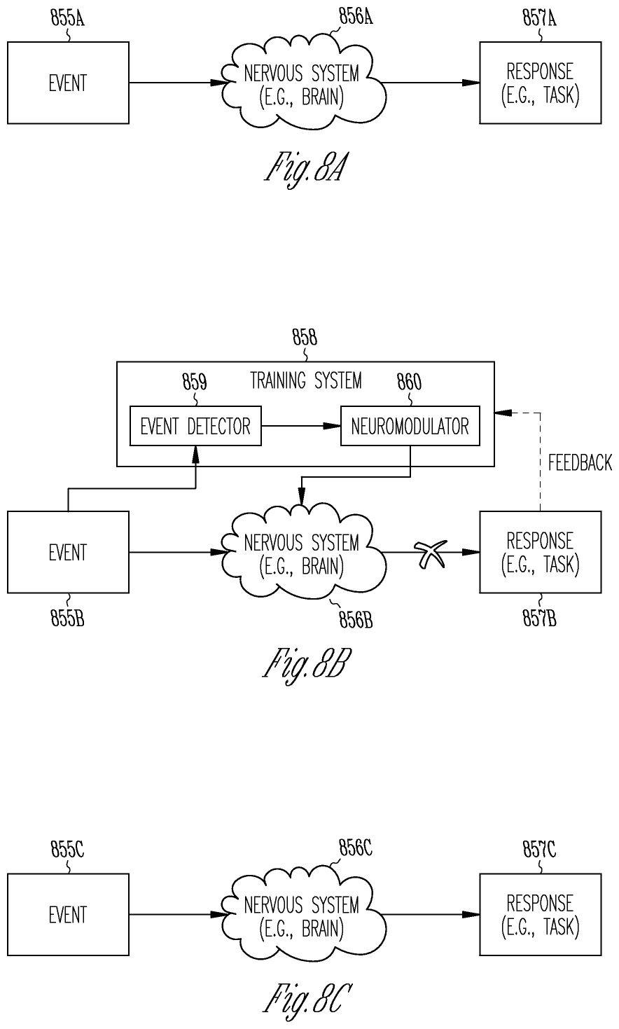

FIG. 7 illustrates, by way of example and not limitation, the electrical therapy-delivery system of FIG. 6 implemented using an IMD. The IMD may include a DBS stimulator. The illustrated system 742 includes an external system 749 that may include at least one programming device. The illustrated external system 749 may include a clinician programmer 704, similar to CP 104 in FIG. 1, configured for use by a clinician to communicate with and program the neuromodulator, and a remote-control device 703, similar to RC 103 in FIG. 1, configured for use by the patient to communicate with and program the neuromodulator. For example, the remote-control device 703 may allow the patient to turn a therapy ON and OFF, change or select programs, adjust patient-programmable parameter(s) of the plurality of modulation parameters, and/or provide inputs used to detect event(s). The patient may use custom external devices to perform specific tasks useful for the event-triggered therapy of that patient. The external devices may communicate directly with the IPG/IMD, or they may communicate with an intermediary device (such as an RC) which in turn communicates with the IPG/IMD. FIG. 7 illustrates an IMD 750, although the monitor and/or therapy device may be an external device such as a wearable device. The external system 749 may include a network of computers, including computer(s) remotely located from the IMD 750 that are capable of communicating via one or more communication networks with the programmer 704 and/or the remote-control device 703. The remotely located computer(s) and the IMD 750 may be configured to communicate with each other via another external device such as the programmer 704 or the remote-control device 703. The remote-control device 703 and/or the programmer 704 may allow a user (e.g., patient, caregiver and/or clinician or rep) to answer questions as part of a data collection process. The external system 749 may include personal devices such as a phone or tablet 751, wearables such as a watch 752, sensor(s) 753 and server(s)s 754. The watch may include sensor(s), such as sensor(s) for detecting activity, motion and/or posture. Other wearable sensor(s) may be configured for use to detect activity, motion and/or posture of the patient. The external system 749 may include, but is not limited to, a phone and/or a tablet. The system 742 may include medical record(s) for the patient and broader patient population(s). The medical record(s) may be stored and accessed using one or more servers (e.g., local or remote servers such as cloud-based servers). The external device may also include device(s) (e.g., app on phone/tablet or a custom device) used by the patient to perform tasks and may also monitor the ability of the patient to perform the task. The external system may be used to process inputs, detect events, analyze the results and/or optimize the training. Processing may be done using cloud computing, fog computing, and/or edge computing. Cloud computing may include a network of devices or servers connected over the Internet. Cloud computing may have very large storage space and processing capabilities. However, cloud computing can have higher latencies. Fog computing occurs physically closer to the end user compared to centralized data centers. The infrastructure of fog computing may connect end devices with central servers in the cloud. Fog computing may provide lower latency for quicker responses and may use other communication technology other than the Internet. Edge computing is done at the device level. The processing for different functions may be distributed over multiple devices and may be distributed over edge computing, fog computing and cloud computing.

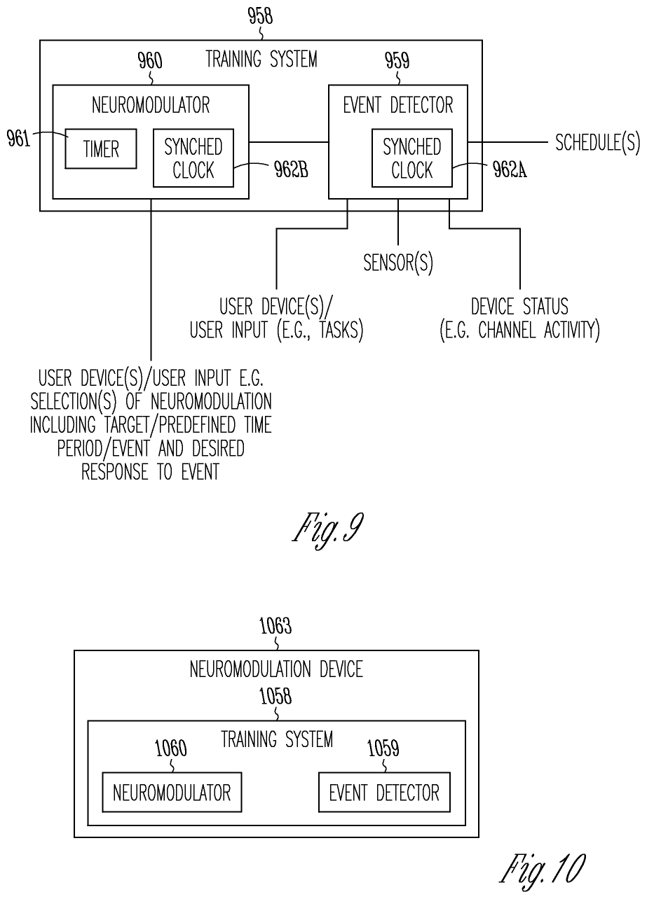

FIGS. 8A-8C illustrate, by way of example and not limitation, a nervous system response, neuromodulation training for training or improving the nervous system based on an event or state within the nervous system (e.g., brain), and a trained or improved nervous system to provide the desired response. With reference to FIG. 8A, the nervous system 856A (e.g., brain) behaves in a manner (e.g., Response/Task 857A) regardless of whether the nervous system is healthy or pathologic. For example, that behavior may be directed (e.g., by asking the subject to perform a task, or by providing an input that is processed and results in an output); or that behavior may happen as a matter of course and is automatically or manually detected (e.g., when a patient feels excessive negative without an apparent rationale).

With reference to FIG. 8B, upon detection (and in some cases processing) of the response to the brain behavior—(i.e., detection of the event), a change in a stimulation may be initiated to reinforce or discourage that brain behavior or pattern. “Event” may refer to things that occur within a task. The event may be the response to a brain behavior (where that behavior can be elicited or naturally occur). Modulating the brain behavior in the context of a properly chosen event can result in improvement of the brain behavior in that context. The training system 858 may include an event detector 859 configured to detect the event 855B, and a neuromodulator 860 configured to deliver neuromodulation energy to the nervous system 856B. The neuromodulation energy may be delivered at a desired time after the event. The response 857B may be used as feedback into the training system. By way of example, the feedback may be used to adjust the timing for delivering the neuromodulation after the detected event. After the training system trains or improves the nervous system, the nervous system 856C provides the desirable response 857C. With reference to FIG. 8C, for example, the trained or improved nervous system 856c may be therapeutic for pathological brain/nervous system or may enhance healthy brain/nervous system) by providing a new or improved response (e.g., task).

Learning generally occurs over relatively long time-scales. In contrast, events may occur over relatively short, task related time-scales. Certain tasks may be particularly good as probes of certain elements or atoms of behavior. For example, some tasks may probe attention and some tasks may probe multi-sensory integration. Different “building blocks of behavior” may together form a complex system of behavior. Various embodiments may incorporate specific tasks and stimulation within those tasks to help “normalize” the behavior of interest.

Various embodiments of the present subject matter attempt to find a particular state in which stimulation may be most efficacious, which may be used to train or obvious cognitive tasks or repetitive things like walking (freezing of gait). Delivery of stimulation at particular points in time with respect to the repetitive thing may be important even if it does not result in any learning per se. An event may punctuate things that may occur within a task. When learning does occur it may occur due to processes that are different from those we control with stimulation. Spacing of training blocks may affect learning and improving success rates.

By way of example, a system may have an external device used to program a stimulator with all of the needed information to respond to an event trigger with at least one response paradigm (e.g., stimulation response parameters—electrodes, amplitude, pulse width, pattern/rate, duration, etc.). A same or different external (e.g., a different external with custom capability for the specific trigger) can simply send a signal to the stimulator to initiate a stimulation response of a particular pre-loaded paradigm. That signal may be very simple and need not include all of the data required to define the stimulation paradigm because that data is preloaded. According to other system embodiments, the external may be configured to send at least part or all of the data used to define the stimulation paradigm.

In some embodiments, the external device may send information to initiate the stimulation change. In some embodiments, the external device may send at least some data that is expected to change or has the potential of being changed from instance to instance (e.g., because that parameter is being optimized). An example of such an optimized parameter may include, but is not limited to, the time after the event. In some embodiments, the external device may send a time stamp of the event. The stimulator may use that data to make sure that it precisely delivers the stimulation change at an appropriate time post event detection.

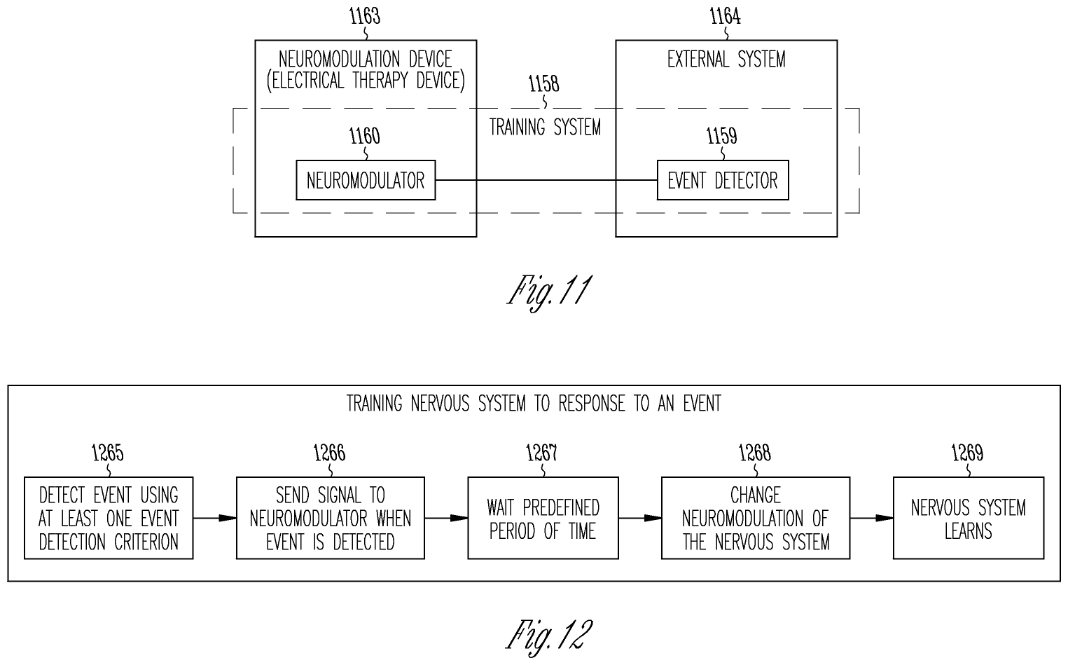

FIG. 9 illustrates, by way of example and not limitation, a training system configured for training the nervous system to respond to an event. The illustrated training system 958 includes a neuromodulator 960 and an event detector 959. The event detector 959 may be configured to detect an event, and the neuromodulator 960 may be configured to respond to the detected event at a desired time after the event. For example, the timer 961 in the neuromodulator 960 may monitor time after the event. Plasticity or adaptation of the neural system appears to occur at different time scales. For example, spike timing dependent plasticity (STDP) is very short on the order of milliseconds. STDP requires pre and post synaptic excitation concordance on the order of 1's to 10's of ms. Plasticity also appears to occur using stimulation within 100's of ms after the trigger (Yttri E A, Dudman J T. Opponent and bidirectional control of movement velocity in the basal ganglia. Nature. 2016 May 19; 533(7603):402-6. doi: 10.1038/nature17639. Epub 2016 May 2. PMID: 27135927; PMCID: PMC4873380; Gittis A H, Yttri E A. Translating Insights From Optogenetics To Therapies For Parkinson's Disease. Curr Opin Biomed Eng. 2018 December; 8:14-19. doi: 10.1016/j.cobme.2018.08.008. Epub 2018 Sep. 12. PMID: 31903441; PMCID: PMC6941740.). This longer timeframe may be due to a network-level change rather than a synapse level change. Network-level changes may operate of different timescales depending on the network, and that trigger-to-stimulation response latency may be a parameter that may be optimized as part of the therapy. The optimal value may even change as the network goes through different states as part of retraining. In order to take advantage of STDP it may be important to have response between less than 100 ms, less than 10 ms or less than 5 ms. The neural system may also have a synaptic scaling phenomena where the strength of connectivity changes more gradually over hours and days.

Further, both the neuromodulator 960 and the event detector 959 may have synched clocks 962A and 962B to accurately time the delivery of the neuromodulation after the event. For example, if the event detector 959 captures a time at which the event happened, that information may be sent to the neuromodulator 960. The neuromodulator 960 may receive a packet of information including a timestamp. When the event detector 959 and the neuromodulator have a synched time, the neuromodulator is able to provide accurate timing for changing neuromodulation after the event. It is noted that there is a limitation on how quickly the detection can take place and how quickly the information packet with the time stamp can be communicated. The event detector 959 may include at least one of a user device configured for use to enter or determine an endpoint of or a period or phase in a task based on time since the task was started or to provide a computer-based information corresponding to a computer-based task, at least one wearable sensor configured to sense a parameter indicative of a status of the task, or at least one sensor attached or integrated into an object used to perform the task. The event detector may be configured to use at least two conditions to detect a state, and to use the detected state as the event. The event detector 959 may detect scheduled event(s) such as may be provided in a schedule, may detect events using sensor(s), may detect device status such as channel activity, or may detect tasks or other user input or interaction with user device(s) such as phones, tablets or laptop computers. The event detector 959 may detect an event, and use other sensed information (e.g., biopotentials) to determine the state of the system to determine whether and what type of reinforcement stimulation to deliver. The event detector 959 may be used to modify the goal of the training stimulation. The signal sent from the event detector to the neuromodulator may include information used by the neuromodulator to determine at least one of a predefined time period for implementing a change to the neuromodulation or information used to change the neuromodulation. The neuromodulator 960 may be configured to change neuromodulation to the nervous system by turning stimulation on or off, changing at least one stimulation value, changing a number of stimulation pulses, changing a stimulation pattern, or changing an electrode fractionalization used to deliver the neuromodulation. The present subject matter may be used to train the nervous system, which includes the brain, the spinal cord, peripheral nerves and ganglia. The neuromodulator 960 may be a deep brain stimulator (DBS) configured for delivering neuromodulation to a brain, a spinal cord stimulator (SCS) configured for delivering neuromodulation to neural targets in or near the spinal cord, or a peripheral nerve stimulator (PNS) configured for delivering neuromodulation to a neural target in the peripheral nervous system. Neurotransmitter respecification is a phenomenon where the neurotransmitter released by a neuron (and the neurotransmitter taken up by the downstream receptors) both change in time from one kind to another. Neurotransmitter respecification has been proposed as a possible mechanism behind depression caused in Northern latitudes in the winter and the recovery from it due to light therapy (e.g., Nicholas Spritzer Lab, School of Biological Sciences at UC San Diego (https://biology.ucsd.edu/research/faculty/nspitzcr)) and it has recently be posited as possibly occurring during STN DBS (Alosaimi, F., Temel, Y., Hescham, S. et al. High-frequency stimulation of the subthalamic nucleus induces a sustained inhibition of serotonergic system via loss of cell phenotype. Sci Rep 12. 14011 (2022). https://doi.org/10.1038/s41598-022-18294-6). Timescales for this are considered to be long (days, weeks). It may be possible to accelerate the timing.

FIG. 10 illustrates, by way of example and not limitation, a neuromodulation device 1063 configured with a training system 1058. Thus, as illustrated, the entire training system 1058, including the neuromodulator 1060 and the event detector 1059, may be part of the same device. Device examples include DBS, SCS or PNS devices. The neuromodulation device may be configured to implement a first process to detect the event and to implement a second process to change the neuromodulation. The first process may be configured to send the signal to the second process. The system may be configured to enable choice of tasks that can optimally probe particular “atoms” of behavior and thereby “optimally train” the nervous system.

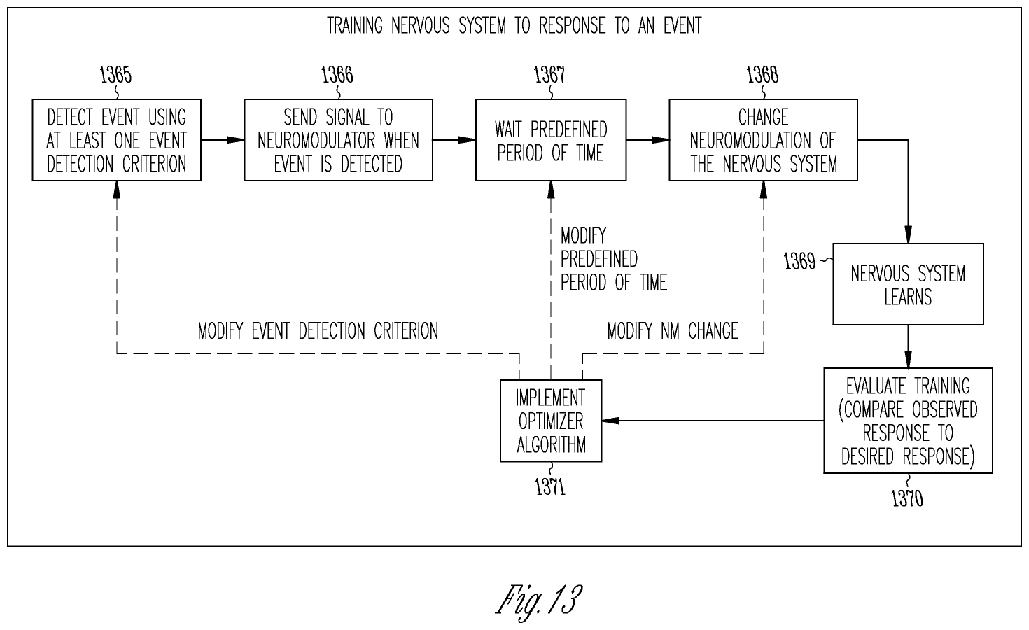

Rather than being contained in the same device, the training system may be distributed across two or more devices. FIG. 11 illustrates, by way of example and not limitation, a training system 1158 distributed among a neuromodulation device 1163 and an external system 1164. Thus, the neuromodulator 1160 may be included in the neuromodulation device 1163, and the event detector 1159 may be included in one or more devices of an external system 1164.

FIG. 12 illustrates, by way of example and not limitation, a process for observing the state of the nervous system by monitoring during a task, and then changing the delivered stimulation to cause a change in the nervous system (i.e., train the nervous system) when the state is ready to be modulated in a favorable way by the stimulation change as determined by an event being triggered. The event trigger is used to know if the nervous system is in a state such that a modulation in stimulation will create a desirable retraining of the nervous system. That retraining may be manifest in nervous system behavior generally, and not only during a prescribed task. The illustrated process may be performed by the training systems of FIG. 9, 10 or 11. The illustrated embodiment includes monitoring to detect an event (or other state information) 1265. In response to the detected event (or other state information, a signal 1266 may be sent to the neuromodulator to perform a neuromodulation-related action (e.g., a neuromodulation pulse, a train of pulses, or a reduction in stimulation that had been ongoing, etc.). The predetermined period of time (or latency) is waited after the signal 1267, and neuromodulation of the nervous system may be changed 1268 after the predetermined period of time, which causes the nervous system to learn at 1269.

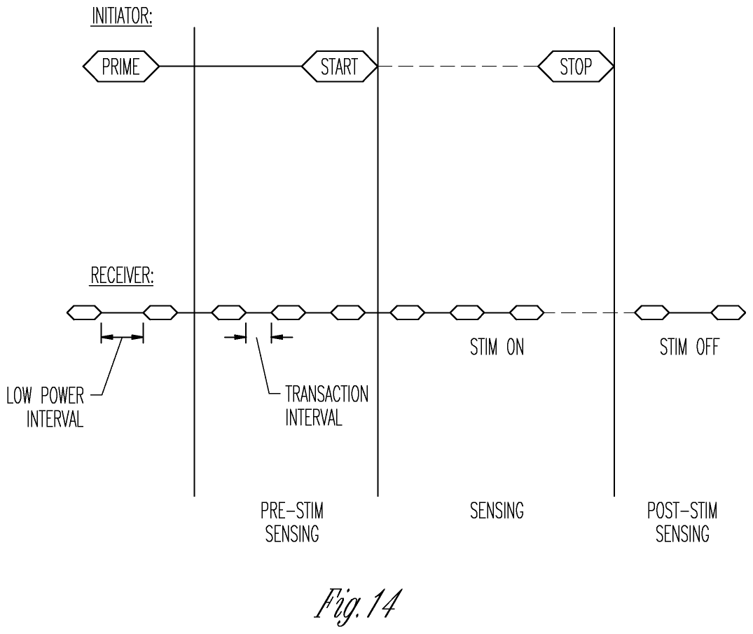

FIG. 13 illustrates, by way of example and not limitation, a process for training a nervous system with an optimizer. The effect on learning may be evaluated by assessing an effect of the stimulation using an appropriate metric. The optimizer may be used to determine if and/or what stimulation parameters should be updated.

Similar to 1265, 1266, 1267, 1268 and 1269 in FIG. 12, the illustrated process includes monitoring to detect an event 1365. Other state information may be used in some embodiments. The illustrated process further includes sending the signal when the event is detected 1366. For example, the signal may be sent to an implantable medical device for the device to perform a neuromodulation-related action such as a change to stimulation. The change to stimulation may be an increase or a decrease in stimulation. By way of example, the neuromodulation-related action may include a reinforcement neural stimulation or an abatement neural stimulation. A reinforcement stimulation is timed and delivered to a location to encourage the observed response (“learned behavior”) of the neural system to the detected event, and an abatement stimulation is timed and delivered to a location to discourage the observed response (“learned behavior”) of the neural system to the detected event. The illustrated process further includes waiting a predefined period of time 1367 and changing the neuromodulation of the nervous system 1368 after the predefined period of time. The nervous system (e.g., brain, spinal cord, or ganglia) learns 1369 function changes in connection with the event used for training. The learned function extends beyond the training period.

The illustrated process identifies capabilities of a system designed to take advantage of plasticity of brain (and other nervous system elements) by modulating brain function in association with a desired event. The process may be implemented as an open loop system. The process may include steps to close the loop and optimize stimulation effectiveness.

The training may be evaluated at 1370. For example, an observed response may be compared against a desired response. The “response” may be a response to a task performed as part of detecting an event 1365 or sending the signal when the event is detected 1366 or may be response to a different task or observation method. Regardless of the response that is being evaluated, the learning is assessed at 1370. An optimizer 1371 may be implemented to optimize the training. Optimizing the training may include one or more of modifying an event detection criteria for detecting the event to trigger sending the signal to the neuromodulator, modifying the predefined period of time after the signal is sent, or modify the neuromodulation before and/or modifying the change in neuromodulation in response to the event. Modifying the event detection criteria may include identifying states that are important for successful training. For example, the optimizer may determine that learning is enhanced if the patient is not in a tired state, not in an anxious state, and/or not in a state of pain. Modifying the predefined period of time may include identifying one or more desired “wait time” less than 100 ms, less than 10 ms or less than 1 ms for appropriate plasticity (e.g., STDP or other types of plasticity) and/or greater than an hour to take advantage of synaptic scaling phenomena or network level changes. Modifying the change in neuromodulation may include changing whether there is neuromodulation before the event is detected and/or changing a change in the modulation in response to the event. Changes to neuromodulation include changes in pulse parameters (amplitude, pulse width, frequency), changes in pulse patterns including a regular (“tonic”) stimulation pulse pattern or an irregular pulse pattern, changes in pulse train or burst parameters such as pulse number in a burst, burst-to-burst timing, and patterns of bursts. Changes to neuromodulation may include changes to the neuromodulation waveform morphology (e.g., rectilinear pulse, multiple rectilinear phases, non-rectilinear pulse shape) and changes to charge recovery (e.g., active, passive, symmetric, asymmetric, intermittent charge recovery pulses). Changes to neuromodulation may include different fractionalization values to change the location and/or shape of the modulation fields. Two or more neuromodulation channels may be used to provide independent modulation of two or more regions. Changes to neuromodulation may include different regions in which to deliver modulation and changing the timing between or among modulation of two or more regions. Changes to neuromodulation may include a reduction in the amount of stimulation or the amount of charge delivered. The modulation may specifically target one or more of STDP and/or synaptic scaling phenomena.

By way of example and not limitation, reinforcement stimulation may be delivered when two or three things happen at the same time or if some things happen (“A” and “B”), but another thing does not happen (“C”). For example, there is a Beta rhythm in Parkinson's disease and a theta rhythm for pain. The state of the patient may be determined using a biopotential such as ECoG or LFP to measure the Theta rhythm. The event detector may not only look at the task that the patient is being trained to do but also may look at the amount of pain the patient is in and use both pieces of information to determine if it is going to send a reinforcement. The state may be determined using sensor(s) and/or user input.

The monitored event may include a task endpoint. In some embodiments, the task endpoint may include a binary outcome such as success/failure. For reward-based tasks, the task endpoint may include the reward the subject received. There may be many types of rewards. For example, possible rewards may include, but are not limited to, stimulation, drug delivery, recognition and metric tracking or trending by an external device. The monitored event may include a performance of a pre-defined task or a change in the performance of a pre-defined task. The pre-defined task may be designed for training purposes.

By way of example and not limitation, the event may be determined using a time since the start of the task, wearable sensors that provide information about status within the task, computer-based information that provides information about status within a computer-based task which may include some cognitive tasks or motor tasks captured on a computer, or sensors integrated into an object used as part of the task. A user, such as a patient, caregiver, clinician, may take an action to manually reflect an event such as a cognitive experience, a mood or affective experience, or may take an action to manually send a trigger in relation to a task. The event may include reaching or encountering a state within or due to a training task and the event signal may be automatically sent when detected or manually sent.

In some embodiments, in addition to event detection, a nervous system state may be determined. The nervous system state may be another input that can affect the system's response to event detection. In some embodiments, state information can include a feature of a measured biopotential. The biopotential may be an evoked response caused by stimulation, sensory input, or a specific task such as a motor task or cognitive task. The biopotentials may be local field potentials (LFPs), electroencephalograms (EEGs), electrocorticography (ECoG), electromyograms (EMGs), electrocardiograms (EKGs), single unit recordings, blood flow measurements (as through near infrared spectroscopy (NIRS) or functional magnetic resonance imaging (fMRI), or others.

A physiological state may be determined by the output of a physiological sensor such as an oxygen monitor, EKG or other heart rate sensor, skin conductance, an accelerometer for detecting posture and/or movement, respiratory rate, blood pressure, some biopotential based signals, external brain state signal (e.g., EEG, magnetoencephalography (MEG), fNIRs, etc.), internal brain state signal ECoG, LFP, single unit record), EMG sensor, symptom measurement (e.g., tremor, rigidity, etc.), temperature sensor, pH sensor, chemical sensor (e.g., neurotransmitter, glucose, steroid, ionic species, etc.), cyclic voltammogram (sometimes used to determine the presence of or to measure one or more aforementioned chemical species), light based signal (e.g., SpO2), or others). The sensor may be used to sense position, velocity, acceleration, a vibration. The sensor output may be processed as part of state. In some embodiments, the sensors could also monitor “behavioral state” directly such as, but not limited to, an eye tracker or a camera. Eye tracker data could be used to track eye movements, both voluntary and involuntary (saccades) while a camera could also be used to track facial expressions. For example, eye movements may be used to infer motor state and facial movements/expressions may infer mood state.

In some embodiments, the state may be defined by multiple signals. For example, the overall state used to trigger an event may be comprised of or based on multiple sub states. Logical operators may be used to combine the multiple signals from sub states to determine if an overall state has been reached. In an embodiment, a multiplexer-like method may be used to combine sub state information. In an embodiment, conditions and logic can be used to integrate information as would be done with a state machine or with a microprocessor. The state may be something that is inferred from performance or a model of performance (but not directly measured) as in Hidden Markov Models.

In some embodiments, the state can be reflected in the status of another stimulation channel. For example, the state of the channel may be a surrogate for the state of a portion of the brain because the channel is modulating a portion of the brain or neural elements. The “surrogate” state can then serve as a trigger for another channel to be activated or deactivated with appropriate timing. An example in the art where this capability can be useful is the modulation of “connectedness” between two brain areas by providing specific timing to strengthen or weaken spike timing dependent plasticity.

Event detection can happen within an IPG, an external device such as a wearable, an external device that is a hub for other external devices (such as a phone or similar that is connected to other wearables typically with wireless communication schemes), and/or in the cloud or in a local computer where processing is done. In some embodiments, one part of the system (e.g., the IPG, or a processing hub) integrates inputs from multiple state identification sources and uses those data to determine an overall state.

With reference to FIG. 12, when specified event(s) is (are) detected or identified, a signal may be sent to an IPG (or other neuromodulation delivery device) to change stimulation (or energy delivery). The “specified event(s)” (and optional states) may be programmable by a clinician user. The “specified event(s)” may be determined a priori, but the user can choose to include or not include them in a therapy. The “specified events” may include a subset of tasks that meet specific criterion(a), which may also be user programmable. The signal may be determined and delivered within the IPG (or other neuromodulation delivery device). The signal may be sent from an external device (e.g., a wearable sensor, a wearable serving as hub, a household instrument or device serving as hub). The signal may be sent as a cellular signal to a local wireless device that in turn sends the signal to the IPG (e.g., via BLE). The signal can be sent via RF, MICS band, Bluetooth or similar schemes, WiFi, electric signals via ionic media, and the like.

The signal sent at 1366 may include event information. In addition to event information, the signal may include timing info (when to effect a change to stimulation or neuromodulation energy), type of change (stimulation information, pattern information, and the like). The system may use rapid communication schemes such that the trigger signal can be sent and received within 1's of milliseconds, or 10's of milliseconds, or 100's of milliseconds, 1's of seconds, 10's of seconds, minutes, or hours such as circadian cycles. For example, Parkinson's Disease and pain therapies may require seconds or minutes. An epilepsy therapy may require milliseconds. With reference to a synapse between a presynaptic neuron and a postsynaptic neuron, Spike Time Dependent Plasticity (STDP) relates to changing a strength of the synapse if the postsynaptic neuron fires an action potential that back propagates to the synapse within a certain latency after having received an excitatory or inhibitory postsynaptic potential delivered from the presynaptic side. There are biological processes that happen at the synapse to strengthen it so that subsequent signals are stronger and are more likely to excite the postsynaptic neuron. For STDP, 1's of milliseconds or lows of 10's of milliseconds may be required (Anders J. Asp, Suelen Lucio Boschen, and J. Luis Lujan An ultra-low frequency spike timing dependent plasticity based approach for treating alcohol use disorder. bioRxiv 2021.09.16.460673; doi: https://doi.org/10.1101/2021.09.16.460673). Dr. Eric Yttri has been researching the coordination of motor, reward and cognitive brain systems (e.g., Yttri E A, Dudman J T; Gittis A H. Yttri E A.).

For task performance triggered based therapy, <200 ms or <300 ms would probably be best (personal communication with Dr. Yttri). Microsecond synching may use precise clocks with regular resynchronization via ionic tissue communication. Predefined sequencies may be used on a granular level. Millisecond level synching may be managed with a combination of internal clocks and BLE for regular synchronization, and 100+ millisecond synching may use real time BLE synchronization. In one embodiment, the IPG may incorporate nerve sensing circuitry to detect the event and may signal the stimulation circuitry to change the stimulation with a predetermined time delay as low as 1's of milliseconds.

In some embodiments, the system may support multiple synch resolutions. In some embodiments, the synch resolution may be manually configurable to specific modes. In some embodiments, the system may automatically determine the synch resolution mode required to be used for a desired event triggered mode and sets the system to the needed mode. In some embodiments, the user may be provided information about the synch resolution mode and the corresponding energy impact, so that the patient or clinician can make appropriate tradeoffs. A higher resolution synch may be required to use event triggering during a specific type of training. The system may temporarily use the higher communications energy mode during training sessions, then manually or automatically revert back to a lower energy communications mode. In some embodiments, the signal that is sent to trigger the event may include or be followed by information indicating how long the change should persist. In some embodiments, the overall “event” may be defined by multiple sub events that are associated with triggers or signals sent to a control system. For example, and event may have a start time sub event (and a start trigger would be sent) and continue for some duration until a sub event stop time is determined (and a stop trigger would be sent). This concept may be implemented with more than two states.

With reference to FIG. 12, neuromodulation of the nervous system may be changed after waiting a predetermined period of time. In some embodiments, the neuromodulation change is an increase in the amount of stimulation by turning stimulation on or increasing a stimulation parameter. In some embodiments, the change in stimulation can include delivery of a relatively small number of pulses. The stimulation may be decreased by turning stimulation OFF (or ON), turning stimulation OFF (or ON) temporarily, or changing one or more stimulation parameters. The stimulation may be changed by changing the pattern of stimulation. For example, the change may be a change to or from a nonregular pattern, from one nonregular pattern to a another nonregular pattern, or from one regular pattern to another regular pattern. In some embodiments, the patterns that are used already reside in memory and are “accessed. In other embodiments, the event trigger signal includes information that contributes to the definition of the pattern. The stimulation may include electrical stimulation or other types of neuromodulation energy such as light, ultrasound, heat/cold, drug delivery (e.g., via a drug delivery pump), and the like. Precise timing may be important in some brain learning processes. Therefore, in some embodiments, the timing of the stimulation change is determined by information provided in the event trigger signal. In some embodiments, the timing information is provided with respect to a clock that is synched with the device that sent the signal. In some embodiments, the timing information includes the time the event happened, where the time to implement the change of stimulation after the event is known to the IPG. In some embodiments, the timing information may include the time to deliver the change of stimulation in accordance with a synched clock. In some embodiments, the change of stimulation may happen as fast as a signal is received, and the timing is managed by the “send” device.

Precise timing may be important in some brain learning processes. In some embodiments, the duration of the change of stimulation may be determined by a user and is programmable a priori. In some embodiments, the duration of the change of stimulation is sent to the control device “along with” the event trigger, which affords for the possibility of variable durations. In some embodiments the duration of the change of stimulation is brief, on the order of a few pulses (milliseconds, 100s of milliseconds, or seconds). In some embodiments the duration is determined by an ON trigger being sent and a subsequent OFF trigger event, where the change of stim is active during the interval. In some embodiments the duration may be calculated by, stored in memory, and auto recalled by the IMD (IPG). Detected event(s) may function as an ON or OFF trigger event. A predetermined duration may be derived from a sensing-based training sequence. The sensed data may be used to calibrate the effective stimulation change duration. Various embodiments may use both ON/OFF triggering events and sensing-based training sequences. A combination of both ON/OFF triggering events and sensing may be used. In some embodiments, the change in stimulation may include manually preprogrammed stimulation parameters including stimulation field, pattern or frequency, number of pulses, pulse width, amplitude.

The change in stimulation may be managed by artificial intelligence (e.g., machine learning). An example is the optimizer 1371 illustrated in FIG. 13. As identified above, machine learning may be used to identify events from which to trigger the reinforcement or abatement neurostimulation or the criterion used to identify the events, may be used to determine the time period(s) to wait after detecting the event(s) and delivering the reinforcement or abatement neurostimulation, and/or the neuromodulation parameters and targeted location(s) for delivering the neurostimulation, including timing relations between stimulating different locations. Machine-learning programs (MLPs), also referred to as machine-learning algorithms or tools, are utilized to perform operations associated with machine learning tasks, such as identifying relationship(s) in the collected data (event(s), event detection criteria, timing between the detected event and neuromodulation change, neuromodulation target(s) and parameter(s). Machine learning is a field of study that gives computers the ability to learn without being explicitly programmed. Machine learning explores the study and construction of algorithms that may learn from existing data (e.g., “training data”) and make predictions about new data. Such machine-learning tools may build a model from example training data in order to make data-driven predictions or decisions expressed as outputs or assessments. The machine-learning algorithms use the training data to find correlations among identified features that affect the outcome. The machine-learning algorithms use features for analyzing the data to generate assessments. A feature is an individual measurable property of the observed phenomenon. In the context of a biological signal, some examples of features may include, but are not limited to, peak(s) such as a minimum peak, a maximum peak as well as local minimum and maximum peaks, a range between peaks, a difference in values for features, a feature change with respect to a baseline, an area under a curve, a curve length, an oscillation frequency, and a rate of decay for peak amplitude. Inflection points in the signal may also be an observable feature of the signal, as an inflection point is a point where the signal changes concavity (e.g., from concave up to concave down, or vice versa), and may be identified by determining where the second derivative of the signal is zero. Detected feature(s) may be partially defined by time (e.g., length of curve over a time duration, area under a curve over a time duration, maximum or minimum peak within a time duration, etc.). The machine-learning algorithms use the training data to find correlations among the identified features that affect the outcome or assessment. With the training data and the identified features, the machine-learning tool is trained. The machine-learning tool appraises the value of the features as they correlate to the training data. The result of the training is the trained machine-learning program. Various machine learning techniques may be used to train models to make predictions based on data fed into the models. During a learning phase, the models are developed against a training dataset of inputs to optimize the models to correctly predict the output for a given input. A training data set may be defined for desired functionality of the closed-loop algorithm and closed loop parameters may be defined for desired functionality of the closed-loop algorithm. Generally, the learning phase may be supervised, semi-supervised, or unsupervised; indicating a decreasing level to which the “correct” outputs are provided in correspondence to the training inputs. In a supervised learning phase, all of the outputs are provided to the model and the model is directed to develop a general rule or algorithm that maps the input to the output. In contrast, in an unsupervised learning phase, the desired output is not provided for the inputs so that the model may develop its own rules to discover relationships within the training dataset. In a semi-supervised learning phase, an incompletely labeled training set is provided, with some of the outputs known and some unknown for the training dataset. Models may be run against a training dataset for several epochs (e.g., iterations), in which the training dataset is repeatedly fed into the model to refine its results. For example, in a supervised learning phase, a model is developed to predict the output for a given set of inputs and is evaluated over several epochs to more reliably provide the output that is specified as corresponding to the given input for the greatest number of inputs for the training dataset. In another example, for an unsupervised learning phase, a model is developed to cluster the dataset into groups and is evaluated over several epochs as to how consistently it places a given input into a given group and how reliably it produces the n desired clusters across each epoch.

Once an epoch is run, the models are evaluated, and the values of their variables are adjusted to attempt to better refine the model in an iterative fashion. In various aspects, the evaluations are biased against false negatives, biased against false positives, or evenly biased with respect to the overall accuracy of the model. The values may be adjusted in several ways depending on the machine learning technique used. For example, in a genetic or evolutionary algorithm, the values for the models that are most successful in predicting the desired outputs are used to develop values for models to use during the subsequent epoch, which may include random variation/mutation to provide additional data points. One of ordinary skill in the art will be familiar with several machine learning algorithms that may be applied with the present disclosure, including linear regression, random forests, decision tree learning, neural networks, deep neural networks, and the like. New data is provided as an input to the trained machine-learning program, and the trained machine-learning program generates the assessment as output. The assessment that is output may be out of an expected range (e.g., anomalous), indicating that remedial action such as retraining of the machine learning algorithm(s) is warranted. The system also may be configured to determine that the new data includes anomalous data with respect to the training data that was used to train the machine-learning program. The detection of new data that is anomalous may trigger remedial action(s) such as, if it is determined that the previously used training data is outdated, retraining the machine learning program using updated training data.

In some embodiments, stimulation may be delivered by a fully implantable system. In other embodiments, stimulation may be delivered by an external stimulator or a combination of an implantable and external stimulation. The stimulator may be connected to other system components so that stimulation is modulated by event triggers. In another embodiment one may consider pairing stimulation modalities to build associations over time so that eventually, the invasive component may be done away with while the noninvasive remains for therapy purposes.

In the following example, sub-state information may be used to define multiple states. By way of example and not limitation, if condition 1=X then do Y while condition Z is present; then do W while condition V is present, and the like. The condition may be a trigger/flag decision tree based on internal and/or external signals, previous history (of patient and device), timer condition (e.g., preset duration) and current state of device and patient). The “Y”, “W” are the set of actions that the stimulator may take specific to the triggering condition. The conditions 1 and Z in this example trigger Y, and condition V triggers W. Condition Z,V can reflect a trigger/flag to exit from actions (Y,W) triggered by triggering conditions (1 and Z in this example).

The system may support pre-set commands and command sequences based on trigger events. For example, the user may preprogram several command sequences on the IPG. When a trigger event occurs, the corresponding sequence may be executed. Sequences may be made so that a full sequence needs to be completed before the device responds to another trigger event (or other event besides ON/OFF). Sequences may have a mandatory phase and an optional phase. In the mandatory phase, once a triggered sequence starts, the mandatory is completed without interruptions. However, the optional phase may be allowed to be interrupted. If a sequence is allowed to be interrupted, a safe interruption or exit sequence could be executed, or return to a safe change state, prior to another event being initiated. There can be different trigger events that can trigger different specific sequences. Mandatory and/or optional phases may be relative to trigger event priorities. For example, interruptions of sequences triggered may only be allowed if a trigger has higher priority than the event that triggered the current running sequence.

With reference to 1369 in FIG. 13, the nervous system is “plastic” and learns in accordance with biological process. Part of the nervous system can be trained (i.e., can “learn”) to be responsive to an arbitrary input (Balint Varkuti, Press & Partners—Ceregate (www.ceregate.com/press-partners/) at Wurzburg conference March 2022). Groups of neurons have a natural ability to “learn” new behavior. Various embodiments of the present subject matter are configured to “teach” or modify nervous system behavior in a way that is therapeutic for a patient or enhances capacity in a useful domain such as a motor task domain (e.g., e.g., Yttri E A, Dudman J T; Gittis A H. Yttri E A.). In principle, this idea could be applied to many neurological disorders, and therefore a system configured to respond to various event triggers will be useful for many neurological disorders.

In patients with major depressive disorder, the brain could be trained to emphasize positive feelings by coupling stimulation to events where patients recall positive events in the patient's life. This activity could be part of therapy sessions where positive experiences are recalled (the task) and upon recalling them (the event) stimulation change is delivered. Conversely, negative experiences could be recalled, and a stimulation parameter could be changed in a different way, or stimulation could not be delivered. In patients with bipolar disorder, a similar therapy exercise could be done, but with the stimulation+therapy designed to train the brain in the direction of emphasizing neutral feeling (to manage highs and lows). In addictive disorders, a similar construct could be followed to teach the brain to be dismissive of the temptation (i.e., reduce attention to the temptation. The therapy session might include framing sensory cues not related to the addictive matter or related to resisting the addictive matter and coupling those with stimulation change designed to strengthen that process in the brain. Similarly, stimulation could be changed in a different way or not delivered when the patient is presented with cues highlighting the addictive substance. The patient might take a pharmaceutical agent (task) and provide manual input to the system (event) to indicate that the drug has been taken. Upon receiving the event trigger, stimulation could be modified to reinforce the action of the drug or train the brain to have a concordant response. In some embodiments, the system accounts for the wash in and wash out of the drug. Other training paradigms can be considered.

In a rehabilitation context, stimulation could be delivered during events where the patient's efforts are most successful (e.g., on target, or suitably fast, etc.), accentuating the successful activity (e.g., e.g., Yttri E A, Dudman J T; Gittis A H, Yttri E A.). This could be useful for stroke relearning, injury rehabilitation such as TBI. In these cases, an activity sensor or group of sensors could be used to measure the successfulness of an attempt. In these cases, processing of task performance (e.g., e.g., Yttri E A, Dudman J T; Gittis A H, Yttri E A.) may be part of the system.

In some embodiments, this could be more general than rehabilitation. For example, reinforcement learning experiments show differences between healthy controls and patients with neuropsychiatric disorders in task performance, differences that show up in both the performance and in how the patients execute tasks relative to healthy controls. Trying to get patients to be more like controls will require a system that monitors and rewards success, but the system may also do it in a manner that incentivizes or motivates the “trajectory” of the patient through the task to look more like a control (or target).

In some embodiments, stimulation may be paired with a reward for successful performance, and an association of stimulation to reward may be built such that eventually stimulation becomes a substitute for the reward. A similar approach may be used to accentuate cognitive performance. For example, during a cognitive training session, the patient may be asked to perform cognitive tasks. Upon “success” event triggering (getting the problems correct, processing quickly, some combination, etc.) the control system delivers stimulation to accentuate performance of the task. This may be useful for patients that are suffering with cognitive decline due to disease (e.g., Alzheimer's, Parkinson's Disease (PD) dementia, other dementia) or injury (e.g., stroke, traumatic brain injury (TBI), etc.).

In some embodiments, the system may be used to train the brain to deemphasize fixation on a certain sensory input or set of inputs. In this case, stimulation may be on generally, and then a cue related to the pathological fixation (the event) may trigger a decrease in stimulation. This approach may be useful for indications such as but not limited to addiction, Tourette's syndrome, tinnitus, and pain.

In the case of pain, stimulation might be used in conjunction with neurofeedback to enhance self-regulation of brain functions by modulating electrical activity of brain areas involved with experiencing pain. Stimulation may be triggered by an analgesic event, like placement of a cold pad, delivery of anesthetic, light therapy, etc. to teach the brain to emphasize “not feeling” or focusing on pain or teach the brain to emphasize feeling relief of pain.

Neuropsychiatric therapies may include stimulation changes in connection with therapy cues (events) to foster positive responses and deemphasize negative responses. In these cases, the delivery of a specific cue may constitute the event and trigger a stimulation change, or the clinician may manually trigger the change based on observing the patient, or the patient may manually trigger the change based upon how they feel.

In the case of overactive bladder (OAB) or urge frequency, a strong void may constitute the event and the stimulation change may be triggered to help the brain emphasize strong voiding with large volumes as a success and to deemphasize weak voids are urges without a void. Alternatively, a strong urge that is ineffective (e.g., flagged by the patient with an external device) may constitute the event, and a change in stimulation (such as a decrease in stimulation) may be triggered to deemphasize activity that results in discomfort due to an ineffective voiding urge. Stimulation changes may be used to manage freezing of gait in Parkinson's patients in an effort to teach the brain not to freeze in certain situations. Balint Varkuti, Press & Partners—Ceregate (www.ceregate.com/press-partners/).

In some embodiments, the effect of the intended “learning” stimulation modulation is measured 1370. For example, in some embodiments, the measurement of task performance (i.e., the effect of the intended therapy) is administered as part of a test that the patient takes. The test may be administered in the clinic and/or may be administered at home. The test may be provided the patient on demand (e.g., through an app on a tablet or phone).

In some embodiments, the tests (and stimulation) may be delivered at specific times of the day. The specific times may be selected to enhance the spacing principle in learning The “spacing principle” in learning refers to enhanced learning when the learning is interspersed with periods of inactivity. Plasticity is enhanced when stimulation is intermittent.

In some embodiments, the measurement of performance may be included as part of the training session (e.g., Dr. Yttri described a mouse performance training scenario where measuring performance was intrinsic in determining event triggering). The performance over time may be tracked and can be shared with the user (e.g., clinician, patient, care giver). The measurement may include measuring motor task performance (e.g., e.g., Yttri E A, Dudman J T; Gittis A H, Yttri E A.) and/or cognitive task performance. A battery of motor tasks may be used to understand (and have the opportunity to optimize) effects of the therapy on multiple aspects of motor behavior (e.g., response time, strength magnitude, strength precision, coordination and/or coordinated responses, etc.), and a battery of cognitive tasks to understand effects of therapy on multiple aspects of cognition (e.g., attention and concentration, types of memory (e.g., declarative, working, procedural, etc.), executive function, processing speed, language and verbal skills). Performance measurements may include perception measurements. Each of senses (sounds, sight, touch, smell, taste) has multiple measurement domains that may be used to measure perception.

In some embodiments, the effect of the intended “learning” stimulation modulation may be measured. An event training tool may be distinct from a measurement tool. In some embodiments, the measurement tool may be integrated with the event training tool. An app is used by the user to acquire measurements (e.g., on a phone, mobile device, computer, etc.). In some embodiments, the measurement may be taken using a wearable device. Examples of a wearable device include an accelerometer-based device, a temperature-based device, a physiological function-based (e.g., heart rate, skin resistance/impedance, a biopotential, etc.), or a stretch-based device. The measurement tool may be a custom tool. The measurement tool (e.g., mobile device, wearable device or custom tool) may be configured with wireless connectivity to provide data to other systems for analysis and/or control (e.g., a control system for controlling stimulation). The control system and measurement tool may be included in the same device such as an external mobile device or custom device.

In some embodiments, the measure may be subjective. The subjective measure may be entered into an app or electronic diary by a patient or caregiver or clinician. Examples of subjective measures may include pain level such as but not limited to visual analog scale or numerical rating scale, mood information such as but not limited to severity, frequency, and episode duration, headache information such as but not limited to severity, frequency, episode duration and attributes of sleep and sleep quality. In some embodiments, the measure may be quantitative. The quantitative measure may be automatically entered into the system. The quantitative measure may be entered manually by a patient, caregiver, or clinician. The measured data may be provided as a report (can be electronic medical records (EMR) compatible) to a clinician, device rep, caregiver, or patient.

In some embodiments, the measured data may be used by an optimizer 1371 which may be used to control the change in stimulation and modify the stimulation for the purpose of optimizing the measured data. The optimizer 1371 may control event detection criteria. The optimizer may be part of the stimulator device. The optimizer 1371 may be in an external device that is not a stimulator. The optimizer may be in a local computer. The optimizer may use cloud computing, fog computing and/or edge computing. The optimizer may use a machine learning, or artificial neural network, or support vector machine, or genetic algorithm, or swarm method or the like to perform optimization. Data from multiple patients (e.g., large patient populations) may be used by the optimizer, or data may be patient specific. By way of example, a patient who struggles with anxiety may require a patient-specific retraining of how to think to avoid an anxiety attack, whereas a motor response may share more similarities with other patients in the larger population. The optimizer may use a simple optimization method such as, but not limited to, gradient descent, simplex, a statistical method, or a binary search. The parameters that the optimizer control and modify may include stim ON/OFF, duration of ON/OFF, frequency or stim pattern related parameters, amplitude, location(s) of stimulation (including adjustment of multiple independent current source parameters), timing of the initiation of the change with respect to the event timing. In some embodiments, the user may set the optimization objective parameters, such as minimize or maximize a measured magnitude, minimize a variability of measurement, reach a target value, minimize or maximize a function selected or defined by the user, the step size to permit, the constraints on variable stimulation parameters (such as those listed in the prior bullet (e.g., frequency selected by optimizer cannot be <X or >Y), or the frequency with which a change is permitted. A graphical or tabular report of the space traversed by the optimizer may be provided. There may be multiple objective functions to manage. For example, the system should be reasonable to use to encourage patient compliance while still gather enough data to optimize the learning. Different techniques may be used, such as optimization algorithms, machine learning algorithms, simple heuristics, statistical approaches, binary searches, and the like. In some embodiments, the optimizer may be permitted to make changes in a manner transparent to the user. In some embodiments, the user (e.g., patient, caregiver, clinician or device rep) is required to acknowledge each change made by the optimizer. In some embodiments, the user can manually change the next set of parameters used in the search.

FIG. 14 illustrates, by way of example and not limitation, timing interaction between an initiator and a receiver to implement a training session. The initiator may initiate the training process and may be central device which may include a neurostimulator device when the neurostimulator device functions as a central device. A receiver may include the neurostimulator when the neurostimulator is not functioning as the central device and various inputs such as sensor(s) or other external devices such as phones or tablets. The initiator may initiate three activities: prime to prepare for the training, a start to the training, and a stop training. The receiver may be operating at a lower power (longer transaction intervals) listening mode to receive a signal from the initiator that a training session (including communication between the receiver and initiator) is desired. Once a session between the initiator and the receiver begins, the receiver may use more power to begin sensing. This sensing may be used to detect event(s). When the event is detected, the initiator may start the neuromodulation change (e.g., Stimulation ON) to provide the reinforcement and/or abatement neuromodulation to one or more neuromodulation targets. The receiver continues to sense while the stimulation is on. The initiator may send a signal to stop the neuromodulation change (e.g., stimulation OFF), after which the receiver may progress to a low-power post-stimulation sensing to determine the effect that the reinforcement and/or abatement neuromodulation had on learning to respond to the detected event(s). Thus, for example, the illustrated timing may be used to train a patient for pain management and to evaluate the outcome of training epochs.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Method examples described herein may be machine or computer-implemented at least in part. Some examples may include a computer-readable medium or machine-readable medium encrypted with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method, comprising:
training or improving a nervous system by responding to an event,
wherein the nervous system is trained by detecting the event using at least one event detection criterion, sending a signal to a neuromodulator when the event is detected, waiting a predefined time period after receiving the signal, and then at an end of the predefined time period changing neuromodulation delivered by the neuromodulator to the nervous system, and
wherein the nervous system has plasticity for learning to respond to the event, and changed neuromodulation at the end of the time period promotes learning by the nervous system to respond to the event.

2. The method of claim 1, wherein the detecting the event includes determining an endpoint of or period or a phase in a task based on time since the task was started, a computer-based information corresponding to a computer-based task, at least one wearable sensor configured to sense a parameter indicative of a status of the task, or at least one sensor attached or integrated into an object used to perform the task.

3. The method of claim 1, wherein the detecting the event includes receiving user input indicative of the event.

4. The method of claim 1, wherein the detecting the event includes detecting a state indicative of the event.

5. The method of claim 4, wherein the detecting the event includes detecting two or more conditions and using the detected two or more conditions to detect the state.

6. The method of claim 4, wherein the detecting the event includes detecting a stimulation channel activity as a surrogate for the event.

7. The method of claim 1, wherein the event includes a trigger event for implementing a command sequence of one or more preset commands, the method further comprising implementing the command sequence in response to the detected event.

8. The method of claim 1, wherein the sending the signal to the neuromodulator includes sending the signal from one process within the neuromodulator used to detect the event to another process within the neuromodulator used to change the neuromodulation.

9. The method of claim 1, wherein the sending the signal includes wirelessly sending the signal to the neuromodulator from another device.

10. The method of claim 1, wherein the signal sent to the neuromodulation includes information used by the neuromodulator to determine at least one of:
the predefined time period; or
information used to change the neuromodulation.

11. The method of claim 1, further comprising determining timing from available timing selections for sending the signal based on the event and a target of the delivered neuromodulation, wherein the available timing selections include two or more of: 1's of milliseconds, 10's of milliseconds, 100's of milliseconds, 1's of seconds, 10's of seconds, minutes or hours.

12. The method of claim 1, wherein the changing the neuromodulation includes:
turning stimulation on or off;
changing at least one stimulation parameter value;
changing a number of stimulation pulses;
changing a stimulation pattern; or
changing an electrode fractionalization used to deliver the neuromodulation.

13. The method of claim 1, wherein the training or the improving the nervous system to respond to the event includes training or improving a brain of a patient to recall events associated with positive feelings or to emphasize neutral feeling.

14. The method of claim 1, further comprising:
evaluating the training or the improving by monitoring an observed response of the nervous system to the event and comparing the observed response to a desired response; and
implementing an optimizer algorithm to improve the training of the nervous system to cause the observed response to the event to be closer to the desired response.

15. The method of claim 14, wherein the implementing the optimizer algorithm includes:
adjusting a set of neuromodulation parameters;
adjusting the predefined period of time for changing the neuromodulation; or
changing one or more of at least one event detection criteria used to detect the event.

16. A non-transitory machine-readable medium including instructions, which when executed by a machine, cause the machine to perform a method comprising:
training or improving a nervous system by responding to an event, including detecting the event using at least one event detection criterion, sending a signal to a neuromodulator when the event is detected, waiting a predefined time period after receiving the signal, and then at the end of the predefined time period changing neuromodulation delivered by the neuromodulator to the nervous system, wherein the nervous system has plasticity for learning to respond to the event, and changed neuromodulation at the end of the time period promotes learning by the nervous system to respond to the event.

17. A system, comprising:
a training system configured for training or improving a nervous system by responding to an event, wherein the training system includes:
a neuromodulator configured for delivering neuromodulation to a nervous system;
an event detector configured for detecting the event and sending a signal to the neuromodulator when the event is detected; and
a timer configured for timing a predefined time period after receiving the signal,
wherein the neuromodulator is configured to change neuromodulation to the nervous system at an end of the time period.

18. The system of claim 17, wherein the neuromodulator and the event detector have synched clocks for use to provide precise timing from a time when the event is detected to a time when the neuromodulation is changed.

19. The system of claim 17, wherein the event detector includes at least one of:
a user device configured for use to enter or determine an endpoint of or a period or a phase in a task based on time since the task was started or to provide a computer-based information corresponding to a computer-based task,
at least one wearable sensor configured to sense a parameter indicative of a status of the task, or
at least one sensor attached or integrated into an object used to perform the task.

20. The system of claim 17, wherein the neuromodulator includes:
a deep brain stimulator (SBS) configured for delivering neuromodulation to a brain;
a spinal cord stimulator (SCS) configured for delivering neuromodulation to neural targets in or near the spinal cord; or
a peripheral nerve stimulator (SCS) configured for delivering neuromodulation to a neural target in a peripheral nervous system.
